(19) United States
(12) Patent Application Publication
(10) Pub. No.: US 2013/0018256 A1
(43) Pub. Date: Jan. 17, 2013

(54) PROBING SYSTEM FOR MEASURING THE DIRECTION AND SPEED OF MUCUS FLOW IN VIVO

(71) Applicants: Hanoch Kislev, Zichron Ya'akov (IL); Itzhak Braverman, Nesher (IL)

(72) Inventors: Hanoch Kislev, Zichron Ya'akov (IL); Itzhak Braverman, Nesher (IL)

(21) Appl. No.: 13/512,094

(22) PCT Filed: Nov. 25, 2011
(86) PCT No.: PCT/IL10/00986

§ 371 Date: Oct. 2, 2012

Related U.S. Application Data
(60) Provisional application No. 61/264,275, filed on Nov. 25, 2009.

(51) Int. Cl. A61B 6/00

(52) U.S. Cl. 600/431

(57) ABSTRACT

The present invention relates to a system and method for measuring the direction and speed of movement of mucus flowing along a ciliary tissue surface, wherein said system comprises: a) a probing unit comprising: dispensing means for controlled seeding of labeled particles into said flowing mucus; probe illumination means for illuminating the mucus flowing over said ciliary tissue surface; optical sensing means for detecting the movement of said labeled particles; optical coupling means for optically coupling said illuminated mucus to said optical sensing means; and b) a control unit comprising at least one illumination source, and means for processing optical or electrical signals received from said optical sensing means and determining the direction and speed of said mucus according to said received signals; c) means for transferring optical or electrical or control signals between said probing unit and said control unit.

### FIELD OF THE INVENTION

The present invention relates generally to a method, system and apparatus for measuring liquid dynamics and more particularly, for diagnosis of in-vivo muco-ciliary dynamics on ciliated tissues.

### BACKGROUND OF THE INVENTION

Ciliary tissue malfunction is responsible for a significant number of diseases and disorders which affect about 30% of the western population. Asthma alone may affect up to 5% of the western population [Fleming et al. BMJ 294:279-283, 1987], and is the most common chronic condition of childhood with between 20% and 25% of all children experiencing wheezing at some point of their life A major cause of Asthma and other respiratory diseases stem from disorders in the respiratory muco-ciliary system.

In human subjects, the ability of ciliary tissue to induce muco-ciliary flow plays an important role in at least five organs: upper respiratory tract, lower respiratory tract (including nose and sinuses), fallopian tubes and eye structure. In these organs the role of the cilia is transport of mucus. For the first four organs, the mucus flow is utilized to propel particles such as cells (including ovum or embryo), debris or foreign particles from the epithelia surface. In the fifth organ the mucus pick the ovum pickup from the fimbria and transport it through the fallopian tubes to the fertilization region and in turn carries the fertilized ovum back to the uterus for the normal embrio growth cycle. Ciliary tissue also moves the mucus to the ventricles of the brain wherein it transports cerebrospinal fluid. Ciliary tissue is also present in Eustachian tubes and connected to middle ear.

The muco-ciliary clearance is defined herein as the ability of the muco-ciliary system to transport a defined fraction of said particles from a specific region, within a given time period.

Nasal muco-ciliary transport is one of the most important local defense mechanisms of the respiratory tract. The organized mucus flow clears debris-laden mucus toward the sinus ostia in well-established flow patterns.

It has been suggested that tissue patterning coupled with mucus flow act in a positive feedback loop to induce effective and controlled mucus flow. It is important to note that during times of stress, such as exercise or infection, the mucu-ciliary dynamics increases so as to accelerate clearance.

Human diseases that disable mucu-ciliary flow, such as primary ciliary dyskinesia, can compromise organ function or the ability to resist pathogens, resulting in recurring respiratory tract infections, sinusitis, otitis, hydrocephaly and infertility.

Although multiple etiologies contribute to the development of rhinosinusitis, a common pathophysiological sequelae is ineffective sinonasal mucociliary clearance, resulting in stasis of sinonasal secretions, with subsequent infection, and persistent inflammation [Bei Chen, J. Shaari, et. al., “Altered sinonasal ciliary dynamics in chronic rhinosinusitis,” Am. J. Rhinol. V 20 p. 325-329 (2006)]

In patients with airway disease, e.g. chronic obstructive pulmonary disease, asthma, cystic fibrosis, and primary cilary dyskinesia, retention of mucus is well known and may be caused by hypersecretion in combination with impaired mucus transport due to reduced ciliary activity. The nasal mucociliary clearance system, (in particular, the ciliary system) is susceptible to damage and could be affected by nasally applied drugs, air-borne particles, pollution, allergens, infection agents as bacteria and viruses. Drugs, allergies, and upper respiratory infections are also known to affect ciliary mobility.

Impairment of muco-ciliary flow in animals may produce infertility by interference with ovum pickup by the fimbria and transport through the fallopian tubes. For this reason, evaluation of the mucu-ciliary dynamics can serve as a viable tool for medical evaluation and treatment of infertile women.

The efficiency of the muco-ciliary clearance system is affected by three main factors: The ciliary dynamics, obstacles to mucus flow, such as inflammation, and the rheological properties of the mucus blanket.

The ciliary tissue activity drives the mucus flow through cyclic motion of the cilia on the majority of ciliary cells. Cilia are tiny hairlike appendages, about 0.25 micrometer in diameter and 3-10 micrometer long, that are built from bundles of parallel microtubules microtubules in very precise patterns. They extend in a “bush like” structure from many kinds of epithelial cells and are found in most animal species and in some lower plants.

Conceptually, the ciliary dynamics can be separated into a low frequency motion, the Metachronal wave frequency—MWF and the high frequency motion, the ciliary beat frequency—CBF. The CBF is the repetition rate of the whip-like motion of a cilium and typically ranges between 3 and 15 Hz. The MWF is the wave-like frequency of cilia moving within a specific area, which may be pictured like a waves propagating in a field of wheat moving in the wind.

The MWF has been studied in many works [see Sanderson, M J, and M A Sleigh, “Ciliary activity of cultured rabbit trachea ciliary beat pattern and metachrony,”. J Cell Sci. V 47: p. 331-347, (1981) and Y. Ohashi et al. “Reduced ciliary action in chronic sinusitis,” Acta Otolaryngol Suppl (Stockh) V 397 p. 3-9 (1983)].

As described above, The MWF indicates the efficiency of the ciliary coordination and in turn affect the efficiency of the muco-cilary system clearance. The MWF is typically determined from the elapsed time of the horizontal propagation of the ciliary wave between defined vectorial positions, [see Wong, L. B., Miller, I. F. and Yeates, D. B. “The nature of mammalian ciliary metachronal wave,” J. Appl. Physiol., V 75(1) p. 458-467 (1993)].

The CBF indicates the elapsed time it takes for the cilia to return back to their starting vertical position, [see Wong, L. B., Miller, I. F. and Yeates, D. B. “Regulation of ciliary beat frequency by autonomic mechanisms in vitro,” J. Appl. Physiol., V 65(4) p. 895-1901 (1988)].

Probing the Ciliary Functioning

The traditional medical evaluation of the respiratory tract, the upper and lower respiratory tract is based mostly on endoscopy, using flexible or rigid endoscopes. The paranasal sinus and nose evaluation are also based on endoscopic examination, preferably using a 4 mm. rigid endoscope connected to a video camera and monitor.

Recent works have suggested that the treatment could become more effective by probing muco-ciliary functioning. Such probing could be conducted by direct mucus flow measurement or by probing the ciliary tissue dynamics. Currently, endoscopes are incapable of providing either data: The endoscopes magnification (typically ×3) is far from sufficient for probing ciliary dynamics. Further, there is no known method which utilize endoscope for measuring mucus flow in-vivo.

One way to probe functioning of ciliary tissue involves taking biopsy of nasal and sinuses tissues and examination of the tissue in vitro, typically for evaluating the CBF. The biopsies needed for micro-photo-oscillographic investigation of specimens are obtained by nasal biopsy or brushing. This method results are not reliable since the samples are taken randomly, the liquid rheologic properties are modified and the tissue sample does not communicate with other tissue sections (thus MWF is not representative). The biopsy based technologies are invasive, require significant expertise and may involve complications such as bacterial contamination. In addition the ciliary tissue is washed prior to testing with fluid whose viscosity is unrelated to the in-vivo mucus viscosity at the sample location. Ciliary testing of nasal brushing samples can show anomalies in cilia anatomy but fail to provide a specific and complete correlation to the clinical condition.

Testing ciliary biopsies in-vitro may be conducted by various microscopy devices and methods. For example detection of back-scattered light (from a single cilia group) described in US patent application 20060256342 to Wong uses mathematical methods for recovery of the CBF from in-vitro samples. This method suffers from all biopsies problems described above.

In contrast, in-vivo probing of ciliary dynamics is considered a challenging task. The sub-micron cilium diameter challenges in-vivo probing of the ciliary dynamics probing, due to the required high magnification and the associated small focal depth. Obtaining a focused image of discrete cilium by a manually held endoscope-microscope is a challenging and time consuming task. Significant image blur is contributed by lateral motion comprising breathing and heartbeat movements of the patient as well as the hand movements of the surgeon. Additional blur is contributed by the relative axial motion between the endoscope and the probed tissue. Thus, The current technologies of auto-focus mechanisms and de-blur algorithms fall short of reconstructing the cilary dynamics.

Muco-Ciliary Probing

A traditional method for measuring muco-ciliary dynamics is the saccharin method in which saccharin is administered to certain point in the nose and the patient is asked to report the appearance of a sweet taste [M. Canciani, E. G. Barlocco, G. Mastella, M. M. De Santi, C. Gardi, G. Lungarellam “The saccharin method for testing mucociliary function in patients suspected of having primary ciliary dyskinesia,” Pediatr. Pulmonol, V 5 p. 210-14 (1988)].

The saccharin test indicates disorder when the transport time is greater than 60 minutes. The Saccharin test can be used to detect severe disorders such as ultrastructural ciliary defects such as cilia dyskinesia. However, the saccharin test which is the current in-vivo screening procedure is characterized by very high false-negative results.

More recently, additional methods have been suggested for probing mucociliary dynamics involving various labeling agents such as radio-isotopes. These tests are more sensitive and objective compared to the Saccharin test. However this group of measurement techniques is time consuming and can only indicate the average clearance rate rather than indicating localized clearance disorders.

Measuring in-vivo mucociliary dynamics using optical methods encounter several problems including: Probe-tissue motion, insufficient endoscope magnification and the mucus flow speed profile over the ciliary tissue.

The mucus flow speed profile comprises an AC component modulated by the CBF frequency, whose magnitude quickly falls from 10 micron to about 100 microns from the ciliary surface. Matsui [1]. Hydrodynamic analyses [2] show that the transition from cyclic flow to steady flow speed occurs within a layer of several tens to one hundred microns, depending on the mucus viscosity, and other hydrodynamic factors.

The mucus flow speed measurements have been reported in several works. ICRP [3] reported a wide range of values depending upon disease, ambient conditions and other factors. For healthy subjects, values of 70 and 92 micron/second for tracheal transport, and 40 micron/second for bronchial transport, respectively. Matsui et. al., [1] measured the speed profile of fluid above the ciliary tissue. They found that the average mucus speed approaches a constant value at height, roughly about 100 microns from the ciliary tissue. These speed values are well correlated with typical response time of the Saccharin test (15-30 minutes) and the transit distance between the Saccharin administering point and the taste sensors position (7-15 cm).

Muco-ciliary disorders are mostly associated with extended clearance time. Since the CBF is not the only parameter affecting the clearance time, probing the mucus dynamics is better correlated to muco-ciliary disorders compared to CBF probing.

Several works (for example see Matsui [1]) report experiments for probing the mucus flow speed in-vitro using the velocimetry technique (for example see Lasne [4]). Typically in biological velocimetry measurements, the labeling particles are fluorescent nanoparticles (FCNs) (Matsui [1]). However, the inventors are not aware of any practical approach to use labeling particles for probing mucus flow in-vivo.

Accordingly, there is a need for non-invasive method and system that can locally probe the muco-ciliary dynamics in-vivo and in real time.

An object of the present invention is to provide an optical system and method operable for probing the motion of mucus on the interior surfaces of a body, in the respiratory tract nose and sinuses, nasopharynx and Eustachian tubes, middle ears, reproductive system and ophthalmic systems.

Another object of the present invention is to provide an imaging system particularly useful in measuring and evaluating muco-ciliary dynamics in a real-time manner, but which may be used in other applications.

Other objects and advantages of the invention will become apparent as the description proceeds.

### SUMMARY OF THE INVENTION

The present invention relates to an in vivo system and method for measuring the speed magnitude and direction of mucus upon tissue comprising ciliary cells, wherein said system comprises a probing unit and a control and analysis unit. The purpose of said probing unit is to probe the mucus flow dynamics on said tissue surface through seeding the mucus by suitable labeling particles. The movement of said labeling particles is recorded and the data is processed for directly determination of the mucus flow along the probed tissue region.

In order to perform its intended function, the probing unit generally comprises:
a. seeding means for seeding labeling particles into the liquid mucus flowing on said tissue surface region;
b. stabilizing means for positioning the optical coupling means at an appropriate fixed location relative to said tissue surface;
c. optical sensing means for detecting said labeling particles motion;
d. optical coupling means for coupling the optical radiation from said labeling particles to said optical sensing means;
e. illumination means for enhancing the labeling particles visibility to said optical sensing means;
f. Data communication means for communicating the data packaged generated by said optical sensing means to said control and analysis unit.

The control and analysis unit generally comprises:
a. a suitable illumination source coupled to the illumination means;
b. processing means for determining the localized speed magnitude and direction of the mucus by analyzing a set of data packages captured from the labeling particles seeded mucus by the optical sensing means.

Optionally, the optical coupling means is imaging optics, the optical sensing means is an electronic imager, and the long slender body comprises means for communicate the image data captured by said electronic imager to said control and analysis unit. Optionally, said illumination means emission intensity is time varying, and said electronic imager capture said image data synchronously with said time varying illumination.

In the first mode of the present invention, the number of dispensed labeling particles introduced into the probed region is small, said probing unit is operable for capturing images comprising the 2-D (tracks) of individual labeling particles and the control and analysis unit is operable for separating at least some of said recorded individual tracks from the set of said captured images.

In the second mode of the present invention, the number of dispensed labeling particles introduced into the probed region is sufficient for generating at least one group (cluster) of labeling particles, said probing unit is operable for recording the migration of labeling particles groups, and the control and analysis unit is operable for analyzing the movement of at least one labeling particles group from a set of data packages generated by said optical sensing means.

Optionally, at least some units of probing means are housed in a maneuverable unit with a long and slender envelope, preferably in the form of an endoscope, operable for navigation within the body.

Preferably some of the probing means are housed in a separate unit, removably attached to the maneuverable unit.

Optionally said removably attached unit comprises at least one mirror which deflects the optical radiation from the illumination means in a direction generally parallel to the probed surface. Optionally said removably attached unit also comprises the labeling particles dispensing means and means for conducting electrical signal from the long slender body to said dispensing means. Optionally said removably attached unit is disposable and attached onto the maneuverable unit before introducing to the subject's body.

Optionally said removably attached unit are operable for coupling at least a significant fraction of the light emitted from the light guiding means into a thin stratum of the flowing mucus on said tissue surface region. Optionally said separate assembly comprising said removably attached unit is operable for illuminating said thin stratum such that said thin stratum coincides with the distal focal plane of said imaging optics.

Optionally said long slender body is stabilized against the probed tissue surface with a flexible means which is released during probing process. Preferably said flexible means is an inflatable balloon. Optionally said inflatable balloon is attached on the long slender body away from its distal tip.

Optionally, said flexible means is combined with said separate assembly. Preferably said flexible means is a toroid inflatable balloon comprising means for dispensing labeling particles into said probed tissue surface region. Optionally said dispensing means comprises a set of foldable legs, each with individual pivot connected to a ring attached to said toroid inflatable balloon. Optionally said combined assembly comprises means for conducting electrical signal from the long slender body to said dispensing means. Optionally the optical sensing means is an optical imager, the coupling optics is imaging optics and the edges of said foldable legs in their activated position define a plane which coincides with the focal plane of said imaging optics. Preferably said combined (flexible and separate) means is disposable. Optionally said labeling particles are FCNs, the illumination source spectrum has a narrow spectral width, and the optical imaging means comprises a dichroic filter which blocks the illumination spectrum to the electronic imager. Optionally said illumination source is pulsed for reducing relative motion and Brownian motion effects.

Optionally said labeling particles are bio-particles comprised within said mucus and the mucus is seeded with suitable substances which enhanced said bio-particles visibility to said optical sensing means. Optionally said labeling particles are scattering particles, said illumination source is coherent and said imaging optics is operable for imaging the holographic patterns generated by interaction of said coherent illumination with said scattering particles onto said electronic imager.

Thus, in a first aspect, the present invention is primarily directed to a system for measuring the direction and the speed of movement of mucus flowing along a ciliary tissue surface, wherein said system comprises:

a) a probing unit comprising means for seeding labeled particles into said flowing mucus, means for illuminating the mucus flowing over said ciliary tissue surface, optical sensing means for sensing the movement of said labeled particles, optical coupling means for optically coupling the optical radiation from the illuminated mucus onto said optical sensing means and means for stabilizing said optical coupling means in a desired position at a set distance from said ciliary tissue surface; and

b) a control and analysis unit comprising an illumination source, means for processing the data packages generated by said optical sensing means and means for receiving said data packages from said optical sensing means.

In certain aspects, the system of the present invention is operable for extracting the CBF from the recorded labeling particles motion history. Optionally the thin mucus stratum illuminated by the removably attached unit is generally at closer distance to the probed tissue surface region in order to facilitate the CBF extraction. Optionally both the mucus flow vector and the CBF are extracted simultaneously during the same probing procedure.

In another aspect, the present invention according to the first mode further comprises a method for performing real-time analysis of mucus flow along a ciliary tissue surface in a mammalian (preferably human) subject comprising the steps of:

(a) Seeding the mucus above said tissue comprising ciliary structures with sufficient number of labeling particles for recording at least one track of individual labeling particle on the electronic imager of the probing unit;

(b) Positioning a magnifying optical imaging assembly, whose focal plane is preferably at close proximity to said surface region wherein said optical imaging assembly images said focal plane onto an optical imager.

(c) Exposing said surface region to a time varying illumination suitable for enhancing visibility said labeling particles to said optical imager;

(d) Capturing multiple images from said optical imager, preferably captured synchronously with said time varying illumination;

(e) Processing said multiple images to extract the 2-D position (or track) histories of at least a portion of said imaged labeling particles so as to extract the mucus flow dynamics parameters from said track histories.

In another aspect, the present invention according to the second mode further comprises a method for performing real-time analysis of mucus flow along a ciliary tissue surface in a mammalian (preferably human) subject comprising the steps of:

(a) Seeding the mucus above said tissue comprising ciliary cells with sufficient number of labeling particles for recording the movement of at least one group (cluster) of labeling particle on the optical sensing means of the probing unit;

(b) Positioning a optical coupling means and optical sensing means at close proximity to said surface region wherein said optical sensing means is coupled to the mucus flowing above said probed surface region.

(c) Exposing said surface region to a time varying illumination suitable for enhancing visibility said labeling particles to said optical sensing means;

(d) Capturing multiple data packages from said optical sensing means, preferably captured synchronously with said time varying illumination;

(e) Processing said multiple data packages to extract the motion history of at least one group of labeling particles so as to extract the mucus flow dynamics parameters from said motion history.

In other aspects, a method for measuring mucus dynamics on accessible surfaces of subject's respiratory system, using the system of the present invention, the method comprises the following steps:
(a) attachment of removably attached probe on the distal end of the maneuverable unit.
(b) Inserting the distal end of the maneuverable unit towards a location of a ciliary tissue to be probed.
(c) Locating the maneuverable unit at close proximity to the desired ciliary tissue region.
(d) Stabilizing the maneuverable unit distal end to the ciliary tissue by releasing said flexible element on the maneuverable unit envelope.
(e) Locally seeding the mucus layer above said ciliary tissue with labeling particles from the dispensing means integrated in the removably attached unit.
(f) Exposing said mucus layer to said modulated light from the removably attached unit, capture images of said mucus region in synchronization with said modulated light sequence and communicate said captured image data to said control and analysis unit.
(g) Process a set of said captured image data for extracting the desired muco-ciliary dynamics parameters.

In certain aspects, the methods of the present invention are used for diagnosing disorders in a subject's upper respiratory system, such as sinusitis. In other aspects, the methods of the present invention are used for diagnosing disorders in a subject's lower respiratory system, such as bronchitis. In other aspects, the methods of the present invention are used to detect mucu-ciliary disorders in the female reproduction system. In other aspects, the methods of the present invention are utilized for detecting damage in the respiratory system due to prolonged exposure to contaminants. In yet other aspects, the methods of the present invention are used to detect mucus flow reversal in a subject's airway system in relation to intubation.

Many other embodiments of the system of the present invention and methods of the present invention are detailed hereinafter.

### BRIEF DESCRIPTION OF THE DRAWINGS

The present invention is illustrated by way of example in the accompanying drawings, in which similar references consistently indicate similar elements and in which:

FIG. 1A illustrates a unit operable for probing flow dynamics within a thin liquid layer above a surface, according to an embodiment of the present invention.

FIG. 1B illustrates a chart in regards with the typical velocity profile above ciliary tissue surface.

FIG. 2A schematically illustrates a system for probing muco-ciliary dynamics on the surface of ciliary tissue region, operating according to an embodiment of the present invention.

FIG. 2B illustrates a magnified view of the system's maneuverable unit distal end.

FIG. 3A schematically illustrates a preferred embodiment of said system's maneuverable unit distal end, with a stiff removably attached unit, operating according to the first mode, and removably attached on it.

FIG. 3B illustrates a magnified view of the removably attached unit.

FIG. 3C illustrates a magnified view of the distal end of the removably attached unit of the present invention.

FIG. 4A illustrates a preferred embodiment of said system's maneuverable unit distal end, with a flexible unit combined with a removably attached unit, at its folded position, removably attached on the distal end, for operation according to the second mode of the present invention.

FIG. 4B depicts a magnified view of the flexible unit at activated position.

FIG. 4C illustrate a magnified view of the flexible unit's distal end region at activated position.

It is noted that the embodiments exemplified in these Figures are not intended to be in scale and are in diagram form to facilitate ease of understanding and description.

### DETAILED DESCRIPTION

The present invention provides methods, system and apparatus for probing micro-fluid dynamics. In particular the methods, system and apparatus are operable for providing in-vivo real time analysis of muco-ciliary dynamics (RT-MCD).

The system of the present invention generally relates to a system for measuring the direction and speed of movement of mucus flowing along a ciliary tissue surface, wherein said system comprises: a) a probing unit comprising:
dispensing means for controlled seeding of labeled particles into said flowing mucus;
probe illumination means for illuminating the mucus flowing over said ciliary tissue surface;
optical sensing means for detecting the movement of said labeled particles;
optical coupling means for optically coupling said illuminated mucus to said optical sensing means; and
b) a control unit comprising at least one illumination source, and means for processing optical or electrical signals received from said optical sensing means and determining the direction and speed of said mucus according to said received signals.
c) means for transferring optical or electrical or control signals between said probing unit and said control unit.

In a preferred embodiment, the system optical sensing means is an optical sensor, preferably an electronic imager.

Preferably, the system further comprising optical light guiding means for guiding the light from the illumination source to the probe illumination means, wherein preferably, the optical light guiding means are fiber optics.

Preferably, the system labeling particles seeded by said dispensing means are selected from the group consisting of: fluorescent nanoparticles, colored particles, directionally reflecting particles, reflecting metal particles and substances which enhance the visibility of natural particles within the mucus to said optical sensing means.

In a preferable embodiment, the system signals from the electronic imager are processed by reconstructing the individual tracks of at least a portion of the labeling particles thus determining the direction and speed of the mucus.

In another preferable embodiment, the system signals from the electronic imager are processed by reconstructing images of a group of labeling particles on the mucus and thus determining the direction and speed of the mucus according to the migrating of the weight center of said group.

Preferably, the system optical coupling means, the electronic imager, the light guiding means, and the means for transferring optical or electrical or control signals are comprised in a maneuverable unit comprising long slender envelope, a distal end, a proximal end; and a handle attached to the proximal end of said elongated envelope.

Preferably, said maneuverable unit comprises two imaging channels, one channel comprising magnifying imaging optics for navigating the maneuverable unit towards the probed ciliary tissue, and the second channel comprises the optical coupling means and optical sensing means for detecting the movement of the labeled particles.

Preferably, said maneuverable unit further comprises: a dispensing means for localized seeding the mucus within, or at close proximity to the imaged region with at least one type of labeling particles; and the probe illumination means.

Preferably, the system probing unit is divided into two distinct units:
a) the maneuverable unit comprising:
   light guiding means for guiding light from the light source in the control and analysis unit to the maneuverable unit distal end;
   optical imager for detecting the movement of said labeled particles;
   optical coupling means for optically coupling said illuminated mucus to said optical sensing means;
b) A removably attached probe unit comprising
   dispensing means for controlled seeding of labeled particles into said flowing mucus;
   probe illumination means for illuminating the mucus flowing over said ciliary tissue surface;
wherein said removably attached probe comprises a view port, and wherein said probe illumination means comprises at least one illumination port, and at least one mirror at large angle to said view port axis, operable for folding the light emitted from the light guiding means and passing through the illumination port, at an angle substantially perpendicular to said view port axis.

Preferably, the maneuverable unit further comprising a filter placed between the optical coupling means and the electronic imager, operable for enhancing the labeling particles contrast vs. background light reaching from the illuminated mucus. Preferable labeling particles are fluorescent particles. A preferable filter is a dichroic filter.

In a preferred embodiment, the system illumination source is a one modulated laser source, which preferably operates at wavelengths selected from the group consisting of: 420 nm violet diode laser, 473 nm laser, diode pumped 532 nm laser, 650 nm diode laser, and 780 nm diode laser.

Preferably, the system means for processing optical or electrical signals received from said optical sensing means further determine the modulation of flow speed induced by the CBF.

Preferably, the system probing unit further comprises a removably attached unit, wherein said removably attached unit is operable for illuminating a mucus stratum at close proximity to the probed ciliary tissue surface, in such way that the CBF modulation of the mucus speed can be also extracted.

Preferably, the system means for processing optical or electrical signals received from said optical sensing means further determine the ratio between the flow speed and the CBF in length units.

Preferably the system further comprises a flexible element installed on said maneuverable unit and adapted for minimizing motion between the probing unit and the probed ciliary tissue surface. Preferably, the flexible element is a toroidal balloon held with its axis substantially parallel to the probing element axis. Preferably, said balloon further comprises at least one labeling particles dispensing means, and electrical leads operable for controlled dispensing of said labeling particles.

Preferably, the system means for delivering optical or electrical signals between said probing unit and said control and analysis unit are electrical cables.

Preferably, the system means for processing optical or electrical signals received from said optical sensor and determining the direction and speed of said mucus according to said received signals is a CPU.

In a preferred embodiment, the system control unit comprises an additional illumination source, and wherein said maneuverable unit comprises a separate optical light guiding means for guiding the light from said illumination source to said first channel.

Preferably, the system dispensing means comprise a well filled with labeling particles, an electronic driven piston and a dispensing cup.

Preferably, the system dispensing means comprise a well filled with labeling particles, an electronic driven piston and a dispensing cup;
the optical sensing means is an electronic imager;
the optical coupling means is imaging optics;
the illumination source is a one modulated laser source;
the means for processing optical or electrical signals received from said electronic imager and determining the direction and speed of said mucus according to said received signals, is a CPU; and
the means for transferring optical or electrical or control signals between said probing unit and said control unit are electric cables.

The method of the present invention is generally directed to a method of performing real-time analysis of mucus flowing on a tissue surface in a subject body, comprising the steps of:
(a) Seeding the mucus above said tissue comprising ciliary structures with a sufficient number of labeling particles;
(b) Positioning the maneuverable unit, wherein the imaging optics focal plane is preferably at close proximity to said surface region wherein said imaging optics images said focal plane onto an optical sensor.
(c) Exposing said surface region to a time varying illumination suitable for enhancing visibility said labeling particles to said electronic imager;
(d) Capturing multiple signal arrays sets from said optical imager, preferably captured synchronously with said time varying illumination;
(e) Processing said multiple signal arrays to extract movement history of at least a portion of said imaged labeling particles so as to extract the mucus flow speed and direction from said movement history.

Preferably, the system labeling particles are scattering particles, said illumination light is coherent light, and the method preferably further comprises the steps of:
(a) Seeding a small liquid region with scattering labeling particles;
(b) Positioning the maneuverable unit with the removably attached unit at close proximity to said liquid region;
(c) Exposing said surface region to modulated laser illumination through the folding mirror of the removably attached unit, operable for inducing holographic pattern around each of said scattering labeling particles;
(d) Capturing multiple signal arrays of said exposed surface region using said optical imager, preferably captured synchronously with said laser modulation;
(e) Processing said multiple frames to identify the loci of interference patterns induce by said scattering particles
(f) Using said loci for reconstructing the tracks of at least a portion of said scattering labeling particles so as to extract the liquid flow parameters from said tracks.

Preferably, the system maneuverable unit further comprises a flexible element attached to the long slender envelope, and the method preferably comprises the steps of:
(a) Inserting the distal end of the maneuverable unit towards a location of a ciliary tissue to be probed.
(b) Locating the maneuverable unit at close proximity to the desired ciliary tissue region.
(c) Stabilizing the maneuverable unit's distal end to the ciliary tissue by releasing said flexible element.
(d) Locally seeding the mucus layer above said ciliary tissue with labeling particles.
(e) Exposing said mucus region to said modulated light and capture images of said mucus region in synchronization with said modulated light sequence.
(f) Process a set of captured image for extracting the desired mucus speed and direction.

Preferably the method ciliary tissue is located on an organ selected from a group consisting of the upper respiratory system, the lower respiratory system, the female reproduction system the eye and brain.

In a preferred embodiment, the invention relates to a system for probing flow dynamics of a liquid flowing on a surface within a small localized region at close proximity to a surface, comprising: a magnifying imaging optics assembly encased in an envelope, an optical imager; a seeding assembly for localized seeding the liquid within, or at close proximity to the imaged region with at least one type of labeling particles; a modulated illumination light source operable for enhancing detection of said labeling particles; means for leading said illumination light to the probed surface region; means for exporting said imager data; means for receiving and processing said imager data for calculating said liquid flow dynamics parameters, wherein said means for leading the illuminating light comprise at least one folding mirror which deflect said illumination light to a direction substantially parallel to said surface.

As defined hereinafter, the term “optical sensor” stands for a 1-D or 2-D (electronic imager) array selected from a group comprising: silicon photo-diode, CCD pixel, CMOS pixel, InGaAs photo-diode or pixel and organic optical sensitive pixel device.

The term “optical coupling means” means one or more optical components which effectively collect optical radiation from a thin region of liquid in front of it and couple it to an optical sensor. The optical coupling means may be selected from a group comprising: imaging optics, focusing optics or guiding optics such as a fiber optics.

The term “probe illumination means” means an optical assembly which effectively couples the light from the light guiding means at least a portion of the light arriving from a light source, possibly via an optical light guiding means, such as a fiber optics arrangement, towards a small volume of the probed liquid.

The term “labeling particles” refers to particles which may form a liquid suspension which may be selected from a group comprising: highly reflective particles such as gold nanoparticles, colored particles with distinct diffuse reflection spectrum, FCNs which fluoresce following exposure to optical radiation, scattering particles capable of producing holographic ring patterns when illuminated by a collimated laser beam. The labeling particles could also include suitable substances operable for attachment or reacting with natural particles suspended in the liquid so as to modify their optical properties and in turn enhance their visibility by the optical sensor.

The term “dispensing means” refers to means operable for controlled release of labeling particles from a reservoir to a small region at close proximity or in the field of view of the coupling optics.

The term “maneuverable unit” means a long and slender envelope comprising an optical coupling means, optical sensing means, light guiding means for the probe illumination means, distal end, proximal end, and a handle attached to the proximal end for maneuvering the maneuverable unit. The maneuverable unit may also comprise, dispensing means for labeling particles, and an additional optical guiding means for illumination during navigation of said maneuverable unit.

The term “removably attached probe” means a unit removably attached to the maneuverable unit and comprises: a view port, at least one illumination port, and at least one mirror at large angle to said view port axis, operable for folding the light passing through the illumination port, at an angle substantially perpendicular to said view port axis. The removably attached unit may comprise a labeling particles dispensing means and may be disposable.

The term “flexible means” refers to a flexible retractable component attached to the maneuverable unit and operable for stabilizing (axially and laterally) the maneuverable unit in respect to the probed surface on which the liquid flows. The flexible means may be selected from the group consisting of: a retractable flexible member such as a leaf spring, an inflatable balloon, a piston, and the means described in U.S. Pat. No. 7,500,971 to Chang. When used in-vivo, the flexible means may be coated with soft and bio-compatible coating to prevent tissue damage when pressed against it.

The present invention is particularly useful for real-time, in vivo measurements of the muco-ciliary dynamics on epithelia, such as of the human fallopian tube, respiratory tract and paranasal sinuses. The present invention is therefore described below with respect to such application, but it will be appreciated, as also be indicated hereinbelow, that the invention could advantageously be employed in other applications such as in Otolaryngology, Pulmonology, Gynecology, Allergy and Ophthalmology.

According to one preferred embodiment of the invention the mucus flow is measured by utilizing labeling particles, wherein the measurement may be carried out in one of the following modes of operation:
i) The first mode of operation is based on the controlled release of very small number of labeling particles in at least one location within, or at close proximity to, the field of view (FOV) of the optical coupling means and tracking the tracks of at least one individual labeling particle; and
ii) The second mode of operation comprises the step of releasing sufficient quantity of labeling particles at close proximity to said FOV and tracking the motion of a labeling particles group consisting at least a portion of the released labeling particles.

Various types of labeling particles could be utilized in both embodiments of the present inventions including: fluorescent nanoparticles (FCNs), spectrally reflecting particles, coherent scattering nanoparticles, and luminescent particles. Alternatively, the particles may be natural (mucus borne) particles whose optical contrast is enhanced, using a suitable substance.

The first mode utilizes velocimetry principles [see for example, Fluorescent particle velocimetry, P. Huang, J. S. Guasto, and K. S. Breuer, J. Fluid Mech. V 566 p. 447 (2006)].

A preferred method for probing muco-ciliary dynamics according to the first mode of the present invention comprises the steps of:

(a) Locally seeding the mucus flowing on the probed region of ciliary tissue within the subject with adequate amount of labeling particles;

(b) Exposing said region to a time-varying illumination sequence from a suitable light source;

(c) Using the optical coupling means and an optical sensor for capturing a set of signals of the said region synchronized with said varying illumination sequence.

(d) Applying data processing operations on said set of images for reconstructing the individual tracks histories, of at least a portion of said labeling particles;

(e) Using said track histories for reconstructing the muco-ciliary dynamics on said probed region.

Various means may be employed to reconstruct the said track histories. For example, the optical coupling means can be imaging optics, the time varying illumination can be pulsed, the optical sensor can be electronic imager and the track history of a labeling particle is obtained by registering the location of said labeling particle in each captured image and the time of capturing.

In some possible preferred embodiments of the invention employing the first mode of operation, the labeling particles are FCNs, the exciting illumination is modulated, and the images capturing is synchronized with at least one modulation frequency. When the mucus is seeded with FCNs and exposed to exciting light, and imaged through a fluorescence imaging assembly, the FCN which flow with the fluid trace visible lines on the images. Indeed FCNs have been employed for fluid flow mapping (velocimetry) in various works, for example Lasne et al., [4] used pulsed illumination for mapping microflow between two adjacent glass plates.

As described hereinabove, using common velocimetry methods for probing in-vivo muco-ciliary dynamics, may encounter difficulties such as:

A) The random motion induced by the operator hand and the subject induces random FOV (field of view) shifts and de-focus challenge standard de-blur algorithms used nowadays.

B) There is no reference plane onto which the image can be focused.

C) The background noise from out-of-focus illuminated FCNs and from the tissue is significant and hard to remove.

Among the problems listed above, the absence of a reference surface for focusing is considered a stumbling block for in-vivo muco-ciliary dynamics probing using an endoscope and labeling particles. Current advances in de-blur image processing enable the sharpening of highly de-focused images. However, in cases where the departure from the focal plane is large or random and the probing time is limited, a standard de-blur algorithm is not capable of sharpening the image to a useful level. For the same reasons, attempting an autofocus control loop is a challenging task due to lack of a reference surface or datum.

The inventors of the present invention unexpectedly discovered that a reference surface for image focusing can be provided by removably attached unit operable for illuminating the FOV within a thin layer substantially parallel to the ciliary tissue surface. Here the fixed position of the removably attached unit in respect to the optical coupling means provides a reference surface and rendering the focusing process unnecessary. The inventors further discovered that supporting said removably attached unit on the probed ciliary tissue region is sufficient for probing muco-ciliary dynamics.

In a preferred embodiment of the present invention, the probing unit is divided into two distinct units:
a) the maneuverable unit comprising:
   light guiding means for guiding light from the light source in the control and analysis unit to the maneuverable unit's distal end.
   optical imager for detecting the movement of said labeled particles;
   optical coupling means for optically coupling said illuminated mucus to said optical sensing means;
b) A removably attached probe unit comprising
   dispensing means for controlled seeding of labeled particles into said flowing mucus;
   probe illumination means for illuminating the mucus flowing over said ciliary tissue surface;
wherein said removably attached probe comprises a view port, and wherein said probe illumination means comprises at least one illumination port, and at least one mirror at large angle to said view port axis, operable for folding the light emitted from the light guiding means and passing through the illumination port, at an angle substantially perpendicular to said view port axis.

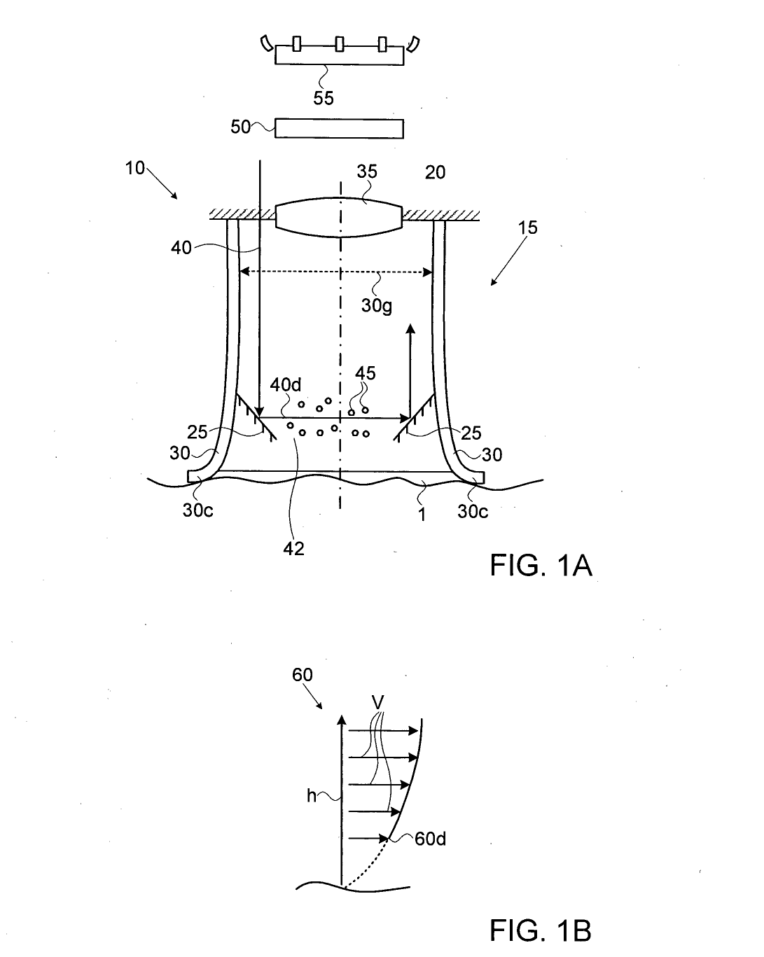

FIG. 1A schematically illustrates the possible structure and function of the removably attached probe according to one preferred embodiment of the present invention. The removably attached unit 10 is operable for illuminating a thin layer of liquid at a pre-determined height above a probed surface 1 with liquid 42 flowing above it. The removably attache unit 10 comprises a support structure 15 employing support legs 30 in the form of a flaring tube, configured to gently lean on surface 1. Optionally, the leading (distal) end of support leg 30 surface may be roughened to provide increased friction on the probed surface 1, and thus reducing possible relative slide. Support structure 15 further comprises at least two tilted mirrors 25, preferably mounted on support legs 30.

When the removably attached unit is attached to the distal end of an envelope comprising an imaging optical coupling means 35 and a suitable illumination source, the illuminating beam 40 is folded by the folding mirrors 25 and in turn illuminates a liquid layer at fixed axial position relative to coupling optics means 35 thereby defining a reference datum. Preferably, the support leg 30 is slotted for minimally affecting the flow speed of the liquid 42 flowing above the probed surface 1. Preferably, the slot(s) opening area may occupy between 10% to 30% of the circumference of leg 30.

The removably attached unit 10 together with optical coupling means 35, imaging sensor and illumination source (not shown) is particularly useful for measuring liquid 42 flow speed at a desired height range above surface 1. The coupling optics means 35 is operable for coupling optical reflection or emission from within the liquid 42 layer illuminated by light beam 40 onto the optical sensor 55 preferably through suitable filter means 50. When the liquid 42 is seeded with labeling particles 45, only the labeling particles 45 within the illuminated liquid 42 layer are visible in the image formed by the optical coupling means onto the optical sensor.

In certain aspects the optical sensor is an electronic imager which is used to capture a set of signals. By comparing the lateral position of discrete labeling particles 45 between consecutive images, one can measure the liquid 42 flow speed within the illuminated liquid 42 region. In other aspects, the migration history of groups of labeling particles is used to measure the illuminated liquid 42 flow speed.

The removably attached unit 10 is further particularly useful for measuring mucus flow speed on a ciliary tissue surface. The deflected light beam 40d illuminates the liquid 42, preferably seeded with labeling particles 45, at a certain height above surface 1. The height of the liquid layer above ciliary surface 1 may generally be about 50 to 300 microns, preferably about 100 microns. At least one light beam 40 from an external illumination source (not shown) illuminates one of the folding mirrors 25 which redirects the beam 40 in a direction generally parallel to ciliary surface 1. A second opposing folding mirror 25 may be provided for redirecting the deflected beam 40d, preferably towards a beam dump.

The use of removably attached unit 10 solves measurement problems associated with measuring mucus flow speed, as will be explained hereinbelow. Diagram 60 shown in FIG. 1B (see for example Lasne [4]) illustrates the mucus flow speed profile above ciliary surface 1, here the abscissa V in FIG. 1B indicate the liquid flow speed, and the ordinate h denotes the height above the ciliary surface 1. The dashed section 65 of the curve located in the proximity (0 to 50 microns) of ciliary surface 1 indicates a region with highly variable and unstable flow speed. By illuminating the liquid layer slightly above said unstable region 65, the removably attached unit 10 enables reliable measurement of the mucus flow speed. ICRF [3] have found that the mucus flow speed above the unstable region is well correlated with the mucus clearance time.

The removably attached unit 10 provides various advantages for muco-ciliary dynamics probing inter alia by: (a) define a reference surface by the tilted mirrors which minimizes axial blur of labeling particles images; (b) Reduce the background noise induced by tissue fluorescence or natural particles scattering; (c) Avoid images of labeling particles above or below the desired probed layer; (d) Reduce lateral blur by stabilizing the line of sight to the probed ciliary tissue using the support legs e) determine the height of the layer being illuminated and in turn coupled to the optical sensor 55, preferably, at height above the unstable speed layer.

Tissue Reference Surface.

In some possible preferred embodiments of the invention, operating the system with the removably attached unit 10 requires the use of a dual channel maneuverable unit, one with lower magnification for viewing the ciliary tissue during navigation and one for recording the labeling particles migration. In other aspects, it is desired to probe the ciliary tissue with a single channel maneuverable unit. In such cases, the use of removably attached unit assembly is complicated and instead an alternative reference surface such as the ciliary tissue should be provided.

In some possible preferred embodiments of the invention, the reference surface is provided by seeding at least a portion of the ciliary tissue surface (e.g., epithelial cells membranes) within the probed region with a different type of labeling particles, for example scattering labeling particles.

In some possible preferred embodiments of the invention, the method for probing muco-ciliary dynamics according to the present invention comprises an additional step of seeding the ciliary tissue region with labeling agent combined with a suitable ligand specific to epithelial cells.

In the absence of removably attached unit, the ciliary tissue does not provide an accurate reference surface. In certain aspects, the coupling optics 35 comprises a auto-focusing mechanism. In some possible preferred embodiments of the invention, the imaging optics assembly is not equipped with an auto-focus mechanism and the axial defocus of the labeling particles tracks is reduced using de-blur algorithms, such as described in Focus Magic software site (2009), http://www.focusmagic.com/.

Scattering Labeling Particles

As mentioned above, the labeling particles can be scattering nanoparticles, and the illumination beam is coherent and the fluid dynamics may be conducted by analyzing the circular holographic interference patterns (rings) generated around each scattering particle by mixing the illuminating laser beam with the laser radiation scattered from said scattering particles (For example see Cheong [5]). The interaction of illuminating coherent and collimated light with the light scattered from such scattering particles induces concentric interference rings in the acquired images around each scattering particles.

In some possible preferred embodiments of the invention, the labeling particles are scattering particles and the illumination light pulse may be extremely short (e.g., between 1 and 100 nano-second, to avoid smearing of the holographic pattern. A suitable algorithm may used to reconstruct each labeling particle track by calculating each labeling particle loci from the center of its ring pattern. Another suitable algorithm tailors a set of loci points for each scattering particle in set of captured images, thereby allowing it to reconstruct the track of each individual particle. Similarly to FCNs, the tracks are used to map the muco-ciliary dynamics.

In certain aspects, the labeling particles are scattering particles and the illumination light is coherent and modulated as sufficiently short pulses. The liquid flow dynamic is analyzed according to the following steps: (a) Illuminating said scattering particles with coherent pulses operable for inducing concentric interference rings around each scattering particles within the illuminated region. (b) Extracting the rings structure around at least some of the scattering particles using a suitable algorithm. (c) Reconstructing at least a fraction of the labeling particle track by connecting the centers of their ring patterns in consecutive image frames. (d) Calculating the liquid speed by dividing the track sections by the period between consecutive images.

The velocimetry method developed by Cheong [5] employs coherent back-lighting for generating holographic patterns around each of said scattering particles. Accordingly the illumination pulses pass through a transparent surface on which the measured liquid flows while the holographic patterns are captured from the front direction.

The inventors of the present invention unexpectedly discovered that by using the removably attached unit of the present invention, one can probe liquid flow on an opaque surface (e.g., ciliary tissue) by illuminating the liquid region with tilted coherent illumination and imaging the probed region, both from the front side. For example the illumination beam is at 45 to 80 deg. to the viewing axis, such that the holographic pattern appears as a set of ellipses. In certain aspects the removably attached unit of the present invention is operable for absorbing the illuminating beam which passes through the probed region, thus enhancing the holographic patterns contrast within the captured images.

In certain aspects, the reflected radiation is attenuated by utilizing the beam properties, for example by using cross polarization techniques. In other aspects, the removably attached unit diverts the incident coherent light at an angle in respect to the viewing axis such that the holographic pattern appears as a set of ellipses. In yet other aspects, the said elliptic holographic patterns are generated such that their short axes substantially parallel to each other.

Muco-Ciliary Dynamics Estimation

Velocimetry using labeling particles is generally conducted according to the following steps [4]:
a) Seeding the probed liquid flowing on a solid surface with labeling particles.
b) Fixing the coupling optics in respect to the surface on which the liquid flows and focusing it on a plane at known distance from the solid surface.
c) Illuminating said seeded liquid with pulsed light and synchronously capturing consecutive image frames of a small FOV within said liquid.
d) Using a suitable algorithm for reconstructing the separate tracks of multiple labeling particles from a set of captured frames.
e) Calculating the liquid speed by dividing the average migration distance of specific labeling particles by the elapsed time between consecutive frames.

For example, the distance in pixels between an individual labeling particle location in consecutive frames is 20 pixels, the pixel size is 5 micron, the coupling optics magnification is ×10 and the elapsed time between consecutive frames is 100 mili-seconds, thus the labeling particle speed is 100 micron/sec.

As described above, the methods of the present invention are operable for estimating the muco-ciliary flow dynamics parameters from a set of images through two modes: (a) First mode: Reconstructing said flow dynamics from discrete labeling particles tracks sections and (b) Second mode: Reconstructing said flow dynamics from the motion of a group of labeling particles.

In some possible preferred embodiments of the invention, probing of muco-ciliary dynamics through the first mode of operation comprises the following steps: (a) Bringing a removably attached unit into contact with the desired ciliary tissue region; (b) locally releasing small amount of labeling particles into, or at close proximity to, at least one location within the probed ciliary tissue region; (c) exposing the probed liquid layer to a time varying sequence of illuminations from a suitable light source (e.g., VPFL-2500 laser from V-GEN Ltd. Ramat-Gan, Israel, combined with suitable second harmonic converter); and capturing multiple images of the illuminated region, said capturing is synchronized with the applied sequence of illuminating beams; (d) processing the captured images in order to extract therefrom tracks of the labeling particles; and (e) estimating the velocity vector histories of multiple individual labeling particles within said captured images by reconstructing the length and direction of extracted track sections along the tracks of the labeling particles.

In some possible preferred embodiments of the invention, each captured image comprises multiple labeling particles image. A suitable algorithm, for example, employing pattern recognition techniques, may be utilized for identifying individual labeling particles in the acquired set of images, for calculating a reconstructable labeling particle history, and for calculating a speed vector for each of said reconstructed track according to the intensity modulation period (e.g., the time between consecutive pulses). The velocity vector data of at least a portion of the labeling particles may be then used for calculating the mucus flow speed and direction Within the probed region.

In some possible preferred embodiments of the invention, the labeling particles are scattering labeling particles, exposing the probed mucus region to pulsed collimated laser beam that induces holographic patterns comprising rings located substantially around each labeling particle. In such embodiment pattern recognition techniques may be employed for identifying in the set of captured images the centers of the rings of light obtained around each scattering labeling particles in the holographic patterns, which are then used for calculating the velocity vectors of each labeling particles and enable the calculation of mucus flow distribution within the probed region.

Probing Mucu-Cilary/Ciliary Dynamics

As described by Smith [2] the mucus speed increases with increasing distance from the ciliary tissue up to several hundred microns. At the same time, the time varying contribution (which is highly correlated to the CBF) of the ciliary dynamics to the mucus speed diminish with increasing distance. Thus at certain distance ranges above the ciliary tissue, the two groups of parameters (average mocus flow and CBF) can be measured simultaneously using the methods of the present invention.

In the most simple scheme, the mucus speed is determined by measuring the apparent migration distance of a labeling particle, as determined by comparing its image position in consecutive frames and dividing the migration distance by the elapsed capturing time between consecutive frames.

In some possible preferred embodiments of the invention, the mucus velocity is determined by reconstructing a selected single labeling particle track. In other aspects, the mucus speed is determined through multiple reconstructed tracks.

In some possible preferred embodiments of the invention, the methods of the present invention carried out according to the first mode of operation are used to probe the ciliary activity parameters, i.e., CBF and MWF. For example, CBF measurements may be conducted by searching a frequency which correlates with the of discrete particles migration vs. time history within the frequency range of CBF. In other possible preferred embodiments of the invention, the removably attached unit of the present invention is adapted to divert at least a portion of the incoming illuminating beams to be slightly angled towards the ciliary tissue surface, such that the beams of illuminating light are directed to a region closer to the ciliary tissue surface at which the CBF contribution to the mucus flow is more significant.

In some possible preferred embodiments of the invention, both the mucus flow parameters and the CBF are measured simultaneously, for example, by calculating the average and time varying distances between adjacent labeling particle image spot. The two data sets may be then manipulated in various ways to deduce various subject disorders. For example high CBF combined with slow mucus flow speed may be an indication of lack of coherence (i.e., weak MWF) between the cilia motion within a ciliary tissue region.

Measurement Performed Using the Second Mode of Operation

A preferred method for probing muco-ciliary dynamics according to the second mode of the present invention comprises the steps of: (a) seeding the mucus flowing on the probed region of ciliary tissue within the subject with labeling particles within at least one location in adequate amount to form at least one distinct group of labeling particles in said mucus; (b) exposing said region to a sequence of time varying illuminating beams of light from a suitable light source; (c) capturing a set of images of the probed region, said capturing is synchronized with the sequence of time modulated illuminating light beams whose intensity and spectrum enhance the labeling particles group visibility to the electronic imager; (d) applying image processing techniques, such as pattern recognition operation on the acquired set of images for reconstructing the average motion of at least one labeling particles group within the probed region; and (e) using the migration history for reconstructing the muco-ciliary dynamics on said probed region.

US patent application No. 2010/0177930 to Dylewski suggests to measure the speed of a fluid moving in a microfluidic channel by locally seeding a liquid flowing in a confined path with labeling particles and sensing a signal related to the labeling particles so as to determine the wavefront (i.e., a small group of labeling particles) location vs. time. The averaged flow speed is estimated by dividing the wavefront migration distance by the time duration between two signal measurements. There however substantial advantages in present invention, for example using the second mode of operation, such as, inter alia: the methods of the present invention collect the labeling particles optical signal only from a defined height above the physical surface and, the methods of the present invention are operable for liquid dynamics reconstruction wherein the liquid flow pattern and direction are unknown a priori.

Labeling Particles and Agent

As described above, the methods of the present invention employ labeling particles which may comprise, but limited to: Colored or highly reflecting particles such as gold nanoparticles, FCNs which are excited by exposure to optical radiation, scattering particles capable of producing holographic ring patterns when illuminated by a collimated laser beam and substances which enhance the visibility of natural particles flowing with the probed liquid, when exposed to suitable illumination.

In some possible preferred embodiments of the invention, the labeling particles used comprise only inert and biocompatible compositions. For example, the labeling particles may be nanodiamonds FCNs (for example see Chang [7]). Preferably, the labeling particles used in the present invention do not interact with the body cells or tissue. The size of FCNs labeling particles may generally be in the range of 10 to 1000 nm, and more preferably between 100 to 300 nm. In other possible preferred embodiment of the invention the labeling particles comprise non-biocompatible composition coated with inert coating, for example quantum dots encapsulated with glass material, preferably through a sol-gel coating process. In other possible preferred embodiment of the invention, the FCNs may be selected from a group comprising red or infrared bands excited quantum dots, fluorescent dye within glass matrix, and the like.

In some possible preferred embodiments of the invention, the labeling particles are FCNs which are fabricated for high excitation brightness, biocompatibility and minimal bleaching, such as nanodiamonds as described by Fu [8]). For example the bleaching energy flux threshold may range between 0.03 to 3 J/cm² during a total period of a few seconds. Preferably, the specific molecular site excitation cross section for the exciting radiation may generally range between 3*10⁻¹⁶ and 3*10⁻¹⁸ cm², and the FCNs overall fluorescent conversion efficiency may generally be, in the range between 1% to 50%.

In some possible preferred embodiments of the invention the FCNs are fabricated for emitting fluorescence in at least two distinguishable spectra (e.g., peaking at 530 and 590 nm), such that two adjacent FCNs may be identified separately in a noisy or blurred image. In other aspects the FCNs size may range the FCNs size may range between 10 to 300 nm.

In some possible preferred embodiments of the invention, the dispensing means systems according to the second mode of operation may carry smaller (e.g., 10 to 100 nm) labeling particles compared to those employed by methods carried out according to the first mode of operation. In other possible preferred embodiment of the invention, the labeling particles are FCNs and their parameters are selected according to a dimensionless merit function expressed as total fluorescence cross section divided by the particle cross section.

Tissue Reference Surface.

In some possible preferred embodiments of the invention it is not desired to use the said removably attached unit for providing a reference datum. In such embodiment, the datum surface is the ciliary tissue surface and used for effective focusing of the optical coupling means onto the probed liquid layer. In such case the labeling particles are mixed with either the fluorescent agent or the attaching labeling particles comprise an attachment reagent which can be selected from: a) antibodies or apatmers specific for epithelial antigens; b) ligands specific for epithelial cell receptors; c) stains specific for epithelial cell nucleic acid; d) an antigen specific for a epithelial cell antibody; e) an analyte specific for an epithelial cell target; or any combination thereof. In other possible preferred embodiments of the invention the said fluorescent molecules have tendency to attach onto epithelial tissue surface, for example using suitable ligands.

Labeling Particles Dispensing Methods

Blume in US Patent application US 2010/0234684 describes an endoscope comprising channels for dispensing biomarkers, a method for releasing such biomarker from the channel opening at the endoscope tip, and methods for analyzing said biomarkers after interacting with certain body fluids or cells.

In some possible preferred embodiments of the invention, the system's maneuverable unit is operable for releasing small number of labeling particles. In certain aspects, the maneuverable unit distal end comprises a channel which stores the labeling particles, an actuator which push a metered volume of suspended nanoparticles and a nozzle which eject the nanoparticles suspension to a predetermined loci at close proximity to the probing FOV.

In some possible preferred embodiments of the invention, the removably attached unit comprises one or more wells, each comprising labeling particles, ejection actuator and a nozzle cup. The ejection mechanism may employ a piezo-actuator, a MEMS cantilever, an electro-transport driver (for dry nanoparticles), or an exploding microbubble means.

Toshiba Ltd. developed a method for injecting nanoparticles into cells suspended within a droplet. The droplet is vibrated on a MEMS membrane at a vibration frequency and amplitude optimized for accelerating the nanoparticles to high speed (see for example [9]). The present invention provides an active well for releasing small and controlled number of nanoparticles, comprising: a well covered with a perforated cup filled with labeling particles suspension, a MEMS membrane and electrical leads. During dispensing, the nanoparticles are accelerated by the membrane vibration and some of them pass through the perforated cup towards the FOV. In other aspects, the well comprises also a movable piston and accordingly, the MEMS motion include frequency components operable for accelerating also said movable piston and assist controlled release of labeling particles, for example by pushing them through a perforated cup.

Illumination Methods

The present invention may utilize various methods of illumination such as, but not limited to: (a) Modulated exciting illumination for inducing fluorescent tracks of the moving FCNs; or (b) Short pulses of coherent light for inducing holographic patterns of scattering labeling particles which move with the mucus. Typically the illumination energy required for scattering particles is in orders of magnitude weaker than that required for FCNs (which is based on the weak fluorescence yield).

In some possible preferred embodiments of the invention, the labeling particles are FCNs, the illumination is pulsed with pulse width and intensity (e.g., 1 micro-second pulse of 10 kW/cm2) sufficient to enable the formation of distinct streaks or points images on the electronic imager when using a dichroic filter. Preferably, the streaks are distinguishable from the background auto-fluorescence of the mucus, carried contaminants and the ciliary tissue. In other possible preferred embodiments of the invention the exciting illumination is modulated such that the labeling particles appear on each captured image as continuous lines sections with pre-determined intensity modulation patten (e.g., 100% intensity short points and 1 to 10% intensity connecting sections).

In some possible preferred embodiments of the invention, there is no physical reference surface and the exciting illumination spectrum comprises two spectral bands, wherein the first spectral band is optimized for exciting the FCNs and the second for exciting the seeded ciliary tissue surface. In other possible preferred embodiments of the invention, the exciting illumination is operable for separate modulation of the first and second spectral bands. In yet other possible preferred embodiment of the invention, the exciting illumination comprises at least one modulated laser source. In further possible preferred embodiments of the invention, the exciting illumination may be selected from a group comprising: Diode pumped 473 nm laser, diode pumped Nd:YAG laser, violet diode laser, 650 nm diode laser, and 780 nm diode laser.

In some possible preferred embodiments of the invention, the exciting illumination modulation period is optimized according to the requirements: a) Detecting FCNs movement (pixels) between consecutive images; and b) minimizing the heating of probed ciliary region. In some possible preferred embodiments of the invention the shortest modulation period may ranges between 0.05 to 0.5 second. The total energy flux incident on the mucus in the FOV may not exceed 1 J/cm2 to avoid undesired tissue photo-chemical effects.

In some possible preferred embodiments of the invention, the exciting illumination sequence includes an initial high flux period for bleaching undesired fluorescence from epithelial or other cells in the probed region (often cellular autofluorescence) or the mucus adjacent to the probed tissue region.

In some possible preferred embodiments of the invention the exciting illumination is periodical according to the estimated CBF of the probed ciliary region. In other possible preferred embodiments of the invention the exciting illumination modulation phase approaches the CBF phase during probing using a lock-in amplifier control loop, preferably using an appropriate real-time algorithm.

Optical Sensor

The optical sensor employed in the systems of the present invention is 1-D or 2-D (electronic imager) array selected from a group comprising: silicon photo-diode, CCD pixel, CMOS pixel, InGaAs photo-diode and organic pixel device. The selection of optimal optical sensor is strongly related to the labeling particles used. For example, a sensitive gray level sensor is optimized for liquid seeded with FCNs labeling particles, where the detection is preferably obtained by using a bandpass filter. RGB imager is optimized for gold labeling particles, where the detection is preferably obtained by dividing the RED signal array by the GREEN signal array. Optimizing the optical sensor for detecting enhanced natural particles depends on the type of enhancement (fluorescent, colored or absorbing).

The number of pixels in the optical may range from 30 to 10 millions and the pixel size may preferably be in the range of 1 to 10 micron. The sensor is preferably externally triggered electronically with a pre-determined shutter time (preferably between 10 micro-second and 1 mili-second). In some possible preferred embodiments of the invention, the optical sensor is an electronic imager having light sensitivity generally in the range of 1 to 100 V/lux×sec (for example see MT9M413 digital image sensor by Aptina Imaging Corp. Singapure). In other possible preferred embodiments of the invention the electronic imager may utilize avalanche photo-sensing imaging array such as in EMCCD (electron multiplying charge coupled device) technology implementations.

Imaging Optics

In some possible preferred embodiment of the present invention, the coupling optics is an imaging optics. In other embodiments the imaging optics has a high magnification typically between ×53 and ×30. In other possible embodiments, the imaging optics has a high numerical aperture for optimizing the amount of light collected from the illuminated labeling particles. In further possible embodiments the imaging optics comprises an optical unit operable for removing low spatial frequency from the generated image, thus increasing the labeling particles visibility. In further possible embodiments, at least one of the imaging optics components is a polymer lens.

In some possible preferred embodiments of the invention, said magnification may be varied by means of one or more movable groups of lenses which may be moved relative to other group optical means and lenses within the optical coupling means. In further possible preferred embodiment of the invention, the size of FOV coupled by the imaging optics may generally be in the range of 30 to 1000 microns, and more preferably between 100 to 300 microns.

In certain aspects, at least a portion of the imaging optics components are comprised in the removably attached unit. In other aspects, at least one of the optical components comprised in the removably attached, unit may be manufactured by processes selected from polymer injection, glass sol-gel process and glass injection.

In some possible preferred embodiments of the invention, the labeling particles are FCNs and the coupling optics comprises a dichroic filter (transmissive or reflective) operable for blocking the exciting illumination from reaching the electronic imager while efficiently transporting the fluorescent radiation to the electronic imager. In further possible preferred embodiment of the invention a dichroic mirror is utilized to deflect the exciting illumination towards the optical sensor.

The selection of the optimal lighting spectrum for probing the muco-ciliary dynamics depends upon various considerations. For example, illuminating mucus seeded with FCNs requires relatively intense short wavelength (typically about 350-550 nm) exciting lighting means, typically in the range of 0.03 to 100 kW/cm², due to the relatively low fluorescent yield. In some possible preferred embodiments of the invention, the localized mucus flow speed can be estimated from a single FCNs reconstructed track.

In contrast, illuminating mucus seeded with scattering labeling particles could employ a laser source operating at longer wavelength (e.g., in the range of 600-1200 nm) considered to induce reduced photo-induced damage. However, detecting the holographic pattern of scattering particle requires higher magnification associated with lower energy flux on the optical sensor.

### EXAMPLE I

A small volume of suspension comprising 140 nm fluorescent nanodiamonds [see Chang [7], is injected into a parallel plates flow cell through which a transparent liquid flows at a maximum speed of 60 microns per second. The liquid flow probing system comprising beam focusing optics, optical imaging assembly with effective magnification of ×10 and numerical aperture of 0.3 and an electronic imager. The electronic imager has a 300×300 array of 4 micron pixels, and its sensitivity is 10 V/lux×second. Next, the probed FOV is exposed to a focused second harmonics 532 nm Nd:YAG laser with a pulse width of 1 micro-second and its flux within the FOV is 10 kW/cm². The implanted NV centers within the nanodiamonds have an average excitation cross section of about 3.1*10⁻¹⁷ cm² and their typical concentration is 200 ppm (Fu [8]. and fluorescent yield of about 30% Hui [10]. The nanodiamond total cross section increase with the nanodiamond size by about 2.7 power due to self relaxation. The pixel of said array generates about 10 mV when exposed to 200 photons of 650 nm. The number of photons collected by the imaging optics onto the nanodiamond image spot is 160 photons per pulse. Assuming that said image spot is contained within a single pixel, respective pixel signal is about 8 mV.

Next, the electronic imager captures images at 20 Hz (i.e., image capture rate of 20 images per second) synchronized with the exciting laser pulses. The distance between image centers of the same nanodiamonds in consecutive captured images is ˜7 pixels. During the measurement, the probed liquid is exposed to an average power density of 20 mW/cm2, where most incident laser power passes through the liquid and reflected to a beam dump.

### Preferred Embodiments

According to one preferred embodiment of the invention a system for examining muco-cilary dynamics on the interior surfaces of a body comprises:

A control and analysis unit comprising a modulated laser source, and a CPU;

A maneuverable unit comprising: a high magnification imaging optics; an electronic imager;

one or more light-guides (e.g. a 500 micron fiber optics) which guide the light from said laser source to the probed mucus layer; means for transferring optical or electrical or control signals with the control and analysis unit; and dispensing means comprising: at least one well filled with labeling particles, an electronic driven piston and a dispensing cup;

According to one preferred embodiment of the invention the maneuverable unit, may comprise: a long (e.g., about 200 to 400 mm) and relatively small diameter (e.g., about 4 to 10 mm) envelope insertable into the body to be examined;

In some possible embodiments, the imaging plane of the imaging optics coincides with the release location of labeling particles.

In some possible preferred embodiments of the invention the labeling particles are FCNs, and a suitable optical filter means is preferably placed between in front of the electronic imager for preventing the reflected probing light from reaching the electronic imager. In other possible preferred embodiments of the invention, the labeling particles are FCNs and the optical filter means is a type of dichroic bandpass filter capable of passing the FCNs peak fluorescence spectral range from the probing light reflected towards the electronic imager.

In some possible preferred embodiments of the invention a removably attached unit operable for folding the illuminating beam substantially parallel to the probed ciliary tissue surface is attached to the distal end of the maneuverable unit. In some possible preferred embodiments of the invention, the removably attached unit is disposable and may comprise one or more dispensing means operable for controlled and localized release of the labeling particles into the probed mucus region. In yet other possible preferred embodiment of the invention, the removably attached unit may comprise an opening which enables viewing the probed mucus within a confined region. In further possible preferred embodiments of the invention, the removably attached unit comprises an integrated slotted leg having one or more elongated slots along its contact surface with the ciliary tissue, for allowing relatively free mucus flow into and from the FOV.

In certain possible preferred embodiments of the invention, the illumination is pulsed and suitable for further reducing the residual blur. For example, for 1 micro-second lighting pulses, ×10 magnification and random relative motion peaking to 1 cm/sec, the labeling particle image spot would move a negligible distance of 0.1 micron.

When maneuvering a rigid maneuverable unit, towards the target ciliary tissue, the operator might impart excessive local pressure at the front end contact point. Acting certain excessive pressure on the epithelial tissue covered by the mucus, even for a few seconds, may induce damage or injury to the delicate ciliary tissue surface. One way to minimize damage to the epithelial tissue is by stabilizing the maneuverable unit on the epithelial tissue using a flexible member such as an inflatable balloon between the maneuverable unit and an opposing tissue surface, as conducted during balloon sinuplasty procedure.

In some possible preferred embodiments of the invention, stabilizing the removably attached unit against the probed ciliary tissue is attained by means of a deployable distal flexible or elastic member, which in its deployed state capable of pushing the removably attached unit away from the probed tissue surface and thereby assist the operator in stabilizing the probe in position. In other possible preferred embodiments of the invention the distal flexible or elastic member is implemented by means of an inflatable flexible/elastic balloon. In further possible preferred embodiments of the invention the inflatable flexible/elastic balloon is filled with sterilized air fed from a small diameter tube connected to, or provided in, the maneuverable unit.

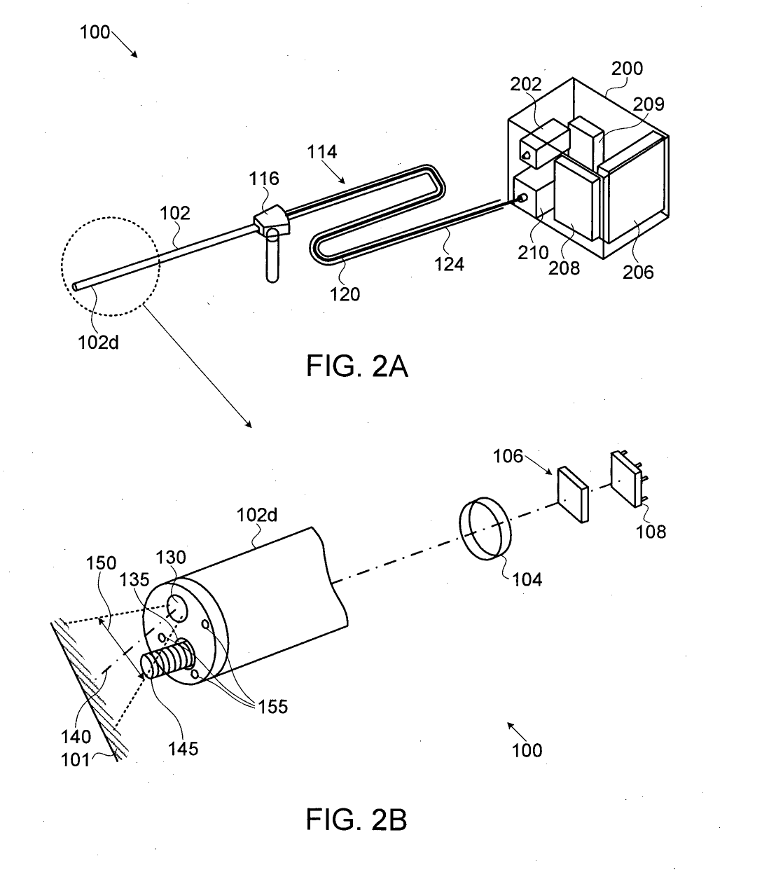

By way of example, FIGS. 2A and 2B schematically illustrate a system 100 for probing muco-ciliary dynamics on a ciliary tissue region 101 within the subject's body, comprising a maneuverable unit 102 in the form of a relatively long and small diameter rigid tube, which distal end 102d is capable of being inserted into the subject's body and maneuvered through body passages thereof. The proximal end 116 of the maneuverable unit 102 is connected via a flexible sleeve 114 to a control unit 200. Flexible sleeve 114 preferably houses an optical light guiding means 120 such as a fiber optics arrangement delivering the light from the control unit 200 to the light guiding means within the maneuverable unit, and suitable electrical cables 124 passing along its length and adapted to deliver optical and electrical signals respectively between maneuverable unit 102 and control unit 200.

The distal end 102d of maneuverable unit 102, shown in more details in FIG. 2B, comprises optical coupling means 104, a filter 106, such as a dichroic filter and an electronic imager 108, front view port 130 with tilted view axis 140 (shown in dot-dashed lines) and means 135 for localized dispensing of labeling particles onto the ciliary tissue surface 101 attached at a known distance from the maneuverable unit distal end 102d. The dispensing means 135 preferably comprises a well filled with labeling particles, an electronic driven piston and a dispensing cup 145 at close proximity to the FOV range 150. The maneuverable unit view axis 140 passing from the optical view port 130 within the FOV range 150. Distal end 102d of maneuverable unit 102 further comprises openings for probe lighting means 155 optically coupled to the light guiding means 120 and adapted to illuminate the tissue region 101.

Control unit 200 preferably comprises a processing means 206 such as a CPU with an electrically linked storage memory for capturing, storing and processing and analyzing the images generated by electronic imager 108. The control unit 200 receives the optical and/or electrical signals from the optical sensor, processes/analyzes the signals and thus determines the direction and speed of the probed mucus. The control unit 200 further comprises two illumination source units: light source 202 for maneuverable unit navigation and light source 210 for illuminating the probed mucus, which may be optically coupled to the same light guiding means 120. The processed captured data may be displayed on a display means 208 (e.g. video monitor).

In some possible preferred embodiments of the invention, the labeling particles are FCNs, the filter 106 is a dichroic filter designed for efficiently blocking the illuminating light reflected back from the probed tissue surface and transmitting the FCNs fluorescence through it to electronic imager 108. Thus, the FOV images captured by the electronic imager 108 mostly comprises the FCNs fluorescence images at high contrast on a dark background. In other possible preferred embodiments of the invention, the light source unit 210 comprises a laser source optically linked to light guiding means 120 whose wavelength is preferably optimized for exciting the FCNs fluorescence.

In some possible preferred embodiments of the invention maneuverable unit 102 is a sterilizable device, and its distal end 102d is sealed to prevent body fluid penetration into the distal end 102 surfaces. In some possible preferred embodiments of the invention, the metal maneuverable unit envelope surfaces are made of a polished metal. In other possible preferred embodiments of the invention maneuverable unit 102 comprises means (for example see a review by Vladkova [11]) for minimizing bacterial biofilm development on its surfaces.

In some possible preferred embodiments of the invention processing means 206 is adapted to display in display means 208 data items selected from a group comprising: images of scene in front of the maneuverable unit imaging optics, one or more calculated mucus speed values, the mucus relative direction in respect to subject's reference axes (e.g., longitudinal and lateral body axes).

A possible procedure utilizing system 100 according to one of the preferred embodiments of the invention will be described in details hereinbelow. After the patient is prepared and ready, the operator introduces maneuverable unit 102 into the patient upper respiratory passages while viewing tissue in front of the maneuverable unit distal end 102d. At a desired point, the operator releases the dispensing means 135 to its deployed state and moves the maneuverable unit distal end 102d until it is at close proximity to the probed ciliary tissue 101. Next, the operator releases flexible means (not shown in the figures) attached to the maneuverable unit distal end 102d into a deployed state, which is used to stabilize the maneuverable unit 102 in position over the probed ciliary tissue 101. Next, the operator releases FCNs particles from the dispensing means 135 and operates control unit 200 to generate a sequence of light pulses from the light source unit 202 and capture a corresponding sequence of FOV images by means of electronic imager 108, which are transferred over the electrical cable 124 to processing means 206. The captured image frames are processed by processing means 206 for calculating the mucus speed magnitude and direction on the probed ciliary tissue 101, as explained in details hereinabove.

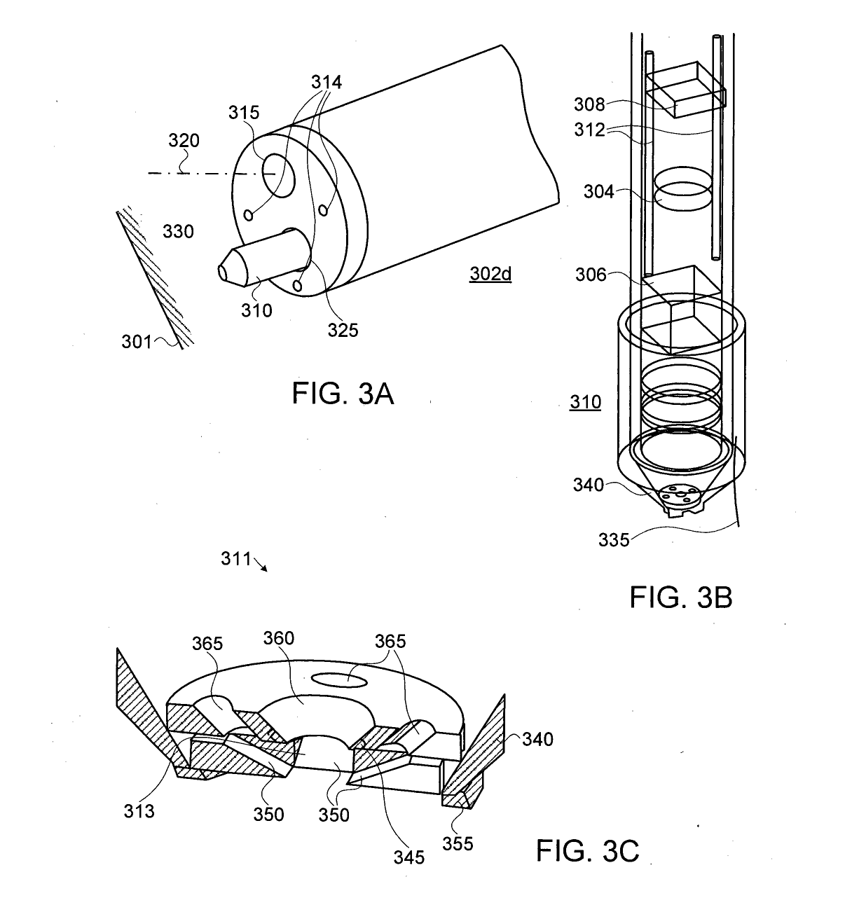

FIG. 3A schematically illustrates a preferred embodiment of a distal end 302d of the maneuverable unit configured for probing muco-ciliary dynamics on a ciliary tissue region 301 within the subject's body. Distal end 302d in this preferred embodiments comprises two imaging channels: (i) the first channel 315 having a parallel view axis 320 and imaging optics (not shown) with generally lower magnification power utilized for navigating the maneuverable unit towards the probed ciliary tissue; and (ii) a probing channel opening 325 which communicates with removably attached unit 310.

With reference to FIG. 3B, removably attached unit 310 is shown attached to the distal end 302d and comprises a conical section 340 tapering distally and a support leg 335 used for stabilizing removably attached unit 310 against the probed ciliary tissue 301. The light from illumination source 210 is delivered via optical light guiding means 120 light guiding means 312, and the beam splitter 306 to the removably attached unit 310. The illuminated region within removably attached unit 310 is imaged by the probing channel through a filter 308, a coupling optics 304, and the clear aperture of the beam splitter 306, all positioned within the probing channel volume.

A sectional magnified view of the distal end of removably attached unit 310 is illustrated in FIG. 3C showing the conical section 340 encompassing an upper chamber 311 whose floor comprises a viewing port 360 and also at least one circumferential illumination port 365. Conical section 340 also encompass a lower chamber 313 comprising at least two beam folding mirrors 350 and a stiff slotted support leg structure 355 to be pressed against the probed ciliary tissue (301) during the probing procedure. The view port 360 provides a view to the mucus volume partially confined in the lower chamber 313. The slots in the support leg structure 355 enable relatively uninterrupted mucus flow into and out of the lower chamber 313.

Optionally the beam splitter 306 comprises a grating operable for coupling the light from the light guiding means 312 to the illumination port(s) 365.

In some possible preferred embodiments of the invention the lower chamber 313 of removably attached unit 310 further comprises one or more dispensing means 345 operable for controlled and localized release of labeling particles responsive to an external triggering means (e.g., mechanical or electrical). Preferably, dispensing means are adapted to direct at least a portion of the released labeling particles to a small localized region adjacent or within the lower chamber 313 of removably attached unit 310.

In some possible preferred embodiments of the invention the dispensing means 345 is triggered by a signal generated in the control unit 200 and delivered to the distal end 302d over electrically conducting lines provided in the flexible sleeve, and in the maneuverable unit (not shown, and over suitable electrically conducting lines provided in (not shown) the removably attached unit 310 and electrically connected to the dispensing means 345.

During probe operation, the light source 210 and the probe is split by the beam splitter 306 into multiple sub-beams. Each sub-beam passes through the respective illumination holes 365, folded by one of the beam folding mirrors 350 and illuminates a thin layer in the lower chamber 313 generally parallel to the probed ciliary tissue surface 301. The illuminated labeling particles in said layer are in turn imaged through the beam splitter 306, and imaging optics 304 onto the electronic imager 308.

In some possible preferred embodiments of the invention, the labeling particles are FCNs and the filter 306 is a suitable dichroic filter capable of blocking at least a portion of the illuminating light scattered within the thin mucus layer.

In some possible preferred embodiments of the invention the removably attached unit 310 is gently pressed against the probed ciliary surface 301 following the release of flexible means (such as an expandable balloon) attached to maneuverable unit 102. Of course, releasing the flexible means also stabilizes (axially and laterally) the probe 310 and focal plane relative to the probed ciliary tissue 301. In other possible preferred embodiments of the invention, the maneuverable unit 102 is stabilized during the measurement by suitable anchoring means operable for reducing relative motion induced by the hand of the operator holding the maneuverable unit.

### EXAMPLE II

The removably attached unit of the present invention comprises a well comprising typically between 10 and 100 nano liter suspension of 10⁸ FCNs per cm³ of the type used in EXAMPLE I above. At this point the system of the present invention with a FOV of 0.3 mm, is manually navigated to a close proximity to the said region surface. Next, the flexible or elastic member is released and in turn, the maneuverable unit is pushed away from an opposing tissue surface. This action gently presses the removably attached unit against the probed ciliary tissue. At that point the well is triggered and release typically 10-100 FCNs, towards the FOV. Next, the FOV is exposed to the pulsed illumination and imaged sequentially onto the electronic imager. Typically 10 to 100 images are captured at a rate of 5 to 20 Hz, in synchronization with the illuminating light pulses. The set of captured images are transferred sequentially to the processing means in the control unit wherein they undergo track reconstruction process. Next the averaged mucus speed in the probed region is extracted from the set of captured images as described in details hereinabove.

A preferred mode of operating, system 100 with distal end embodiment 302d illustrated in FIG. 3A will be now described. The maneuverable unit distal end 302d with removably attached unit 310 attached to it is inserted through the upper respiratory tract of the patient's body. The operator preferably utilizes a navigation illuminating light applied via apertures 314. At that point, the maneuverable unit distal end 302d images the illuminated scene in front of it onto the electronic imager 308. The operator navigates the maneuverable unit distal end 302d, until the removably attached unit is at close proximity to the ciliary tissue region 301 to be probed.

Next, the operator releases the flexible or elastic member which in turn presses the removably attached unit against the target tissue until the maneuverable unit is substantially supported between the ciliary tissue region 301 and a substantially opposing tissue surface. Shortly thereafter, the system 100 is switched to probing mode. Initially, control signals are produced in the control unit 200 and transferred over electrically conducting lines within the removably attached unit 310 to the dispensing means 345 for inducing the localized and controlled release of labeling particles from at least one dispensing means 345 to the partially confined mucus. Upon their release, the labeling particles are swept with the partially confined mucus including the FOV volume.

Simultaneously, the probe illumination source 210 is turned on, delivering modulated light to the mucus within the removably attached unit 310. The electronic imager 108 captures images of the confined mucus in synchronization with the modulated light source sequence. The labeling particles 360 track histories are utilized for estimating the mucus flow speed and direction.

When the mucus flow measurement in the selected region is completed, the light source is turned off, the flexible or elastic member is at least partially deflated (or folded), and the operator turns on again the navigating light and moves the distal end 302d with the removably attached unit 310 toward another target ciliary tissue region 301 and repeats the probing procedure as described above.

In one possible embodiment of the present invention, the removably attached unit further comprises a movable shutter operable for temporarily blocking the mucus flow within the FOV. Various shutter structures may be considered, including numerous movable a similar to the vanes of a shutter within a film camera. The shutter may be placed approximately parallel to the leg 335 surface so as to decouple the ciliary tissue from the mucus layer within the FOV.

In certain aspects, the removably attached unit 310 is stabilized on the ciliary tissue 301 whereas the mucus flow into and out of the lower chamber 313 is blocked by a suitable mechanical shutter (not shown). Next, the following events occur simultaneously: a) the labeling particles 360 are released from the dispensing means 345 towards the FOV; b) the mechanical shutter is opened, enabling mucus flow into from the lower chamber 313, and c) the modulated light from the light source 210 passes through the illumination hole 365 and illuminates the mucus within the FOV. In turn, the shear stress imparted by the moving cilia slowly increases the speed of the mucus within the FOV until reaching an equilibrium speed V₀. The labeling particle migration is recorded by the imager 108 and the processing means 206 extract the speed history from the tracks and calculate recovery parameters such as the elapsed time from opening the shutter until the mucus speed exceeds V₀(1−e).

### Embodiment Utilizing Scattering Particles

As described in EXAMPLE I, employing the system of the present invention with FCNs labeling particles requires relatively intense green illumination (e.g., 532 nm) pulses, even when using highly efficient nanodiamonds. Although the tissue heating is negligible, in particular when using the removably attached unit, some undesired photo-chemical reactions may be induced on the surface of certain probed tissue e.g., ophthalmic tissue).

In certain aspects, it is desired to use the system of the present invention with longer wavelength excitation lightings which is known to induce minimal undesired photo-chemical reactions. As described above, scattering particles may generate holographic pattern when exposed to coherent illumination of wide range of the optical spectrum.

As described above, the use of scattering particles for velocimetry has been demonstrated by Cheong [5]. Theoretically, The holographic rings intensity is comparable to the illuminating beam intensity. This scheme appears attractive since it may enable dramatically reduced illuminating beam intensity. Practically, the rings are detected by collecting small angle scattering radiation from natural small nanoparticles in the probed liquid. The small angle scattering represents only a small fraction of the illuminating beam intensity. Thus, viewing the holographic rings requires the blocking of the illuminating beam with a suitable optical scheme.

However, in the scheme employed by Cheong [5], the probed liquid is back-illuminated and the holographic rings are observed from the front. Clearly, the materialization of a back illumination scheme for measuring mucus on ciliary tissue appears challenging.

When illuminating a liquid seeded with scattering labeling particles with beam whose direction deviates slightly from perpendicular to the view axis, the illuminating beam is easily blocked for example, by a beam dump and the holographic rings can be viewed as a set of ellipses. However in such scheme, the imaging optics collects large angle scattered radiation, and in turn the rings observed intensity is significantly reduced.

In some possible preferred embodiments of the invention, the removably attached unit is utilized for generating the holographic rings. The folding mirror(s) angle is set for illuminating the mucus at optimized large angle to the view axis. In other aspects, the holographic rings contrast is enhanced by simultaneously seeding the mucus with suitable small scattering nanoparticles optimized for enhancing the large angle scattering intensity. In some possible preferred embodiments of the invention, the holographic rings pattern have a few micron size and their detection requires higher magnification levels compared to the respective magnification useful for FCNs. Typical magnification level useful for detecting the ellipses of said interference pattern ranges between ×10 to ×50 leading to a reduced depth of field. In other aspects, near-IR laser illumination is used for generating the holographic pattern and in turn eliminating possible photo-damage to the epithelial tissue.

A preferred embodiment of the present invention suitable for operating with scattering labeling particles preferably comprises: a maneuverable unit; a removably attached unit; and a control unit comprising a coherent light source, processing means and memory means.

In some possible preferred embodiments of the invention, an angular filter (i.e., filter which reject certain angular incidence range of the radiation reaching the optics the is integrated in the imaging optics for blocking light unrelated to the holographic pattern, thus enhancing the holographic pattern over the background radiation. In other possible preferred embodiments of the invention the scattering particles are nanoparticles fabricated for enhanced holographic pattern generation.

### Embodiments for the Second Mode

As described above, probing the muco-ciliary dynamics according to the second mode looks at the migration of particle groups rather than the tracks of discrete particles. Thus, there is no need to separate the individual tracks, and the particle group image can be easily distinguished from the background noise (e.g., fluorescence when using FCNs). In turn, one can consider optical imaging assembly with lower magnification and lower numerical aperture, resulting in higher focal depth. In addition, group of FCNs generate more fluorescence, thus reducing the required illumination power density in comparison to system operating according to the first mode.

In some possible preferred embodiments of the invention the system operated according to the second mode of operation utilizes the removably attached unit described above. Typically, the amount of released labeling particles is relatively higher compared to the amount released when operating the system in the first mode of operation.

In certain aspects, the use of system of the present invention with a removably attached unit and a flexible element complicates the system operation. The inventors of the present invention unexpectedly discovered that it is possible to stabilize the maneuverable unit distal end (102d or 302d) in respect to the probed ciliary tissue and provide a reference surface using a single flexible unit, which may be disposable.

In certain aspects, said flexible unit comprises: an inflatable balloon and means for locally seeding the mucus at known position in respect to the distal end 102d. In other cases, introducing a flexible element between the maneuverable unit distal end 302d and the probed ciliary tissue may reduce the reference surface position accuracy in respect to the imaging optics. In such case, there is an advantage for seeding the mucus with larger number of labeling particles, according to the second mode of operation.

In yet other aspects, said flexible means is a disposable unit, removably attached on the distal end 102d. In other aspects, said flexible means comprises a dispensing means for labeling particles. In yet other aspects, the dispensing means release labeling particles at a know axial position in respect to distal end 102d when said flexible means is activated.

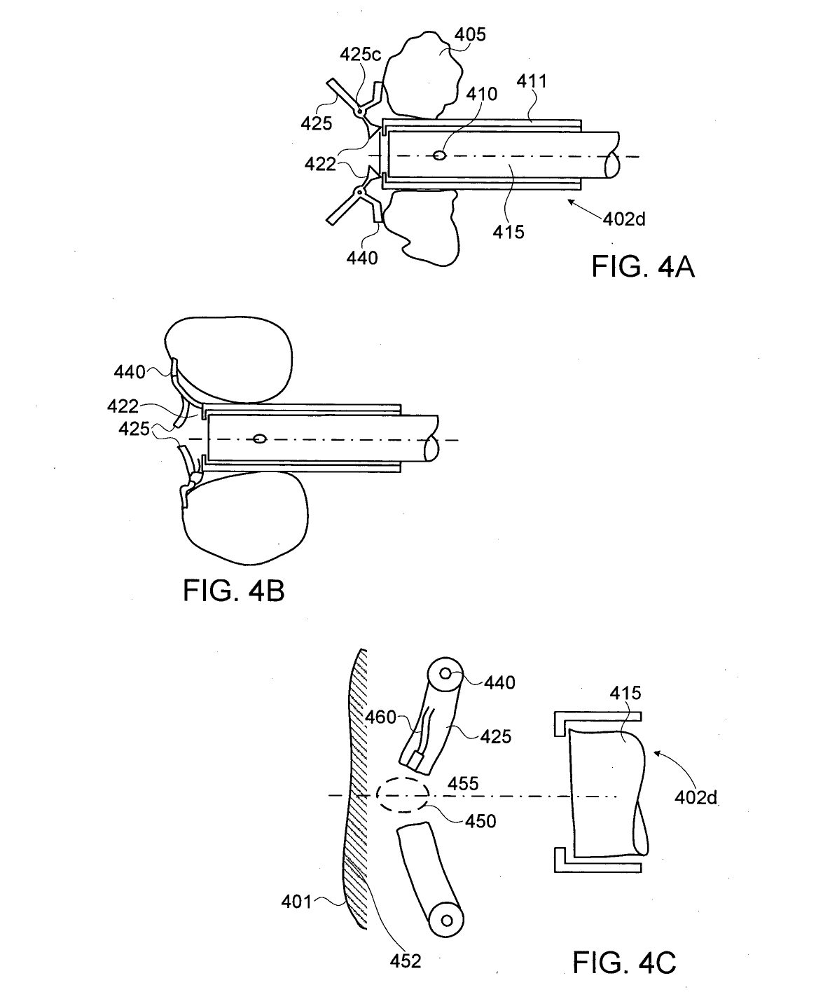

FIGS. 4A, 4B and 4C schematically illustrate a yet another preferred embodiment of an maneuverable unit distal end 402d of system 100 operating according to the second mode and comprising: a flexible means combined with a removably attached probe 411, removably attached on the maneuverable unit distal end 402d and comprising: a view tube section 415, connected to a toroidal balloon 405, which can be filled through a port 410 provided in the distal end 402d. Multiple retractable circumferential cage legs 425, each comprising a cantilever 425c and connected with a pivot (a) to an arm 422 extending from the circumferential distal end of view tube 415 and (b) to a pivot on ring 440 attached to toroidal balloon 405.

FIG. 4A and 4B depicts the preferably disposable flexible means 411, in its deflated and activated states, respectively. In the deflated state, the ring 440 is closer to the maneuverable unit distal end 402d and through the pivot pull retractable legs 425 is brought into close proximity with the toroidal balloon surface. Inflating toroidal balloon 405 pushes the ring 440 away from the distal end 402d and in turn rotate legs 425 to their activated position where they form a cage with toroidal balloon 405 and view tube 415.

FIG. 4C illustrates a magnified view of the disposable flexible means 411 at its activated state. The extended legs 425 partially confine a defined volume around the FOV 450, within the mucus layer 452. At least one of the legs 425 is equipped with a dispensing means 455 comprising labeling particles. The dispensing means 455 is triggered by an electrical signal which passes from the control unit 200 via the flexible cable 122, contacts on the maneuverable unit distal end 402d, contacts on the balloon 405, and electrical leads 460 within the cage legs 425. Since the legs 425 are connected to view tube 415 which is supported on the maneuverable unit distal end 402d, the mucus 452 is seeded at known axial and position in respect to the distal end 402d. In turn, the labeling particles propagate mainly within the field of view 450.

In some possible preferred embodiments of the invention, the distal surface of the toroidal balloon 405 comprises miniature slotted ribs (not shown) operable for minimizing the interference to the mucus flow into and out of the probed FOV 450, when the balloon 405 is pressed against the probed ciliary tissue 401 region. In other aspects, the ribs on the distal surface of the balloon 405 determine the axial location of the FOV 450 in respect to the ciliary surface 401 when the balloon 405 is pressed against the probed ciliary surface, preferably between 50 and 500 microns above the ciliary surface 401.

A preferred mode of operating of system 100 with maneuverable unit distal end 402d, according to the second mode will be now described. The operator introduces the distal end 402d into the upper respiratory tract of the subject, and navigate the maneuverable unit distal end 402d towards the ciliary tissue 401 to be probed, until the toroidal balloon 405 touches the desired ciliary tissue region 401.

Next, the operator inflates the toroidal balloon 405 until the distal end 402d is substantially stabilized between the probed ciliary surface 401 and an opposing tissue surface. Following the balloon 405 inflation, the legs 425 partially confine a mucus volume around the FOV 450 at close proximity to the probed ciliary tissue. Next, control signals from the control unit 200 are delivered to electrical leads 460 for dispensing labeling particles in dispensing means 455. Upon triggering, suitable amount of labeling particles are locally released to the mucus in or at close proximity to the FOV region 450. At the same time the FOV 450 is illuminated with modulated light emerging from the maneuverable unit distal end 402d.

Upon their release the labeling particles are swept as a group with the mucus partially confined between the legs 425. Thus, the weighted center of the swept labeling particles group(s) appears migrating on the set of images with the mucus 452 flow direction. From the said set of captured images, one can reconstruct the labeling particles migration history and in turn the mucus flow speed and direction.

When the mucus flow probing is completed, the modulated illumination is turned off, the balloon 405 is partially deflated, and the cage legs 425 are rotated to the folded position. Next, the operator move the distal end 402d toward another ciliary tissue region 401 and repeat the procedure using system 100 as described above.

In some possible preferred embodiments of the invention, the magnification of imaging optics module 104 is preferably between ×5 and ×15, and sufficient for reconstructing the migration of group of labeling particles, rather than detecting the migration of discrete labeling particles used in the first mode of operation.

Applications

The methods and systems of the present invention are operable for real time analysis of muco-ciliary dynamics (RT-MCD) using endoscopic assessment of a ciliary tissue regions including but not limited to: nose and paranasal sinuses, surfaces respiratory epithel of the respiratory tract, nose and sinuses, nasopharynx and Eustachian tubes, middle ears, reproductive system and ophthalmic tissues.

Accurate diagnosis is made more difficult by the fact that the asthma response and patient metabolic involved are not the same in all patients. Clearly a need exist for an accurate diagnostic method for identifying those asthmatic patients who would benefit from administration of certain types of pharmacological agents.

The system of the present invention may be employed for conducting accurate diagnostic of asthmatic patients, and benefit them by selecting more optimized administration of preferred types of pharmacological agents. Hyper reactive airways as asthma usually produce more mucus and cough by irritation of the bronchi and construction resulting in dyspnea.

When cells in the airways generate mucus at excessive rate, blockages frequently occur in the airway system. The mucus builds up, narrowing the airways. This in turn reduces air flow rate into and out of the lungs. The lining of the airways may also become inflamed, and the muscle surrounding the airways may constrict. These problems cause the airways to narrow even more leading to an escalating asthma attack.

Cilia may be also damaged as a result of tobacco smoking. Smoking harms the cilia functioning and in turn reduces the CBF. Accordingly, the cilia are unable to sweep mucus and particles away and as a result, when cilia don't work, mucus and other irritating substances build up in the airways. Some of the cilia are destroyed. Cigarette smoke also causes the lungs to make more mucus than normal.

Electron microscopy from asthma patients revealed that their mucus plugs consist of moderately electron-dense floccular material containing degenerate epithelial cells, macrophages and cell fragments. The luminal surfaces of ciliated cells showed cytoplasmic blebs and abnormal cilia. Mast cells in various stages of degranulation were scattered between bronchial epithelial cells. The subepithelial hyaline layer, commonly referred to as “thickened basement membrane”, consisted of collagen fibrils in plexiform arrangement. The basement membrane proper appeared intact.

These above described changes, particularly the presence of mast cells and subepithelial collagen deposits, were also found in autopsy samples. This combined light and electron microscopic study shows possibly irreversible changes may be present in the lungs of patients with severe bronchial asthma, even when they are asymptomatic.

Thus, early mucu-ciliary diagnosis followed by suitable treatment may avoid such irreversible changes. For example, detecting abnormal muco-ciliary and CBF data in asthma patients using the system of the present invention, may lead the doctor to use expectorants and bronchodilators in the treatment.

As described above, enhanced CBF and degraded mocu-ciliary dynamics may be detected simultaneously using the system of the present invention. Detecting such effect may lead the doctor to use pulsatile irrigation. Studies indicate that the pulsatile method is effective, because the steady pulsation at the correct frequency restores the normal synchronous beat of the nasal/sinus cilia.

In certain aspects, the system of the present invention is used for diagnosing muco-ciliary related disorders such as: sinusitis, eustachian tube malfunction, serious otitis media and hearing loss. Sinusitis may be induced by one of these conditions: significantly reduced CBF, increased mucus production due to common colds and allergies, and bacterial contamination. A very slow muco-ciliary flow due to excessive mucus builds up, indicates potential for harming bacteria growth. In other aspects, the system of the present invention is useful for the evaluation of the severity of chronic sinusitis and nasal polyposis, preferably together with CT diagnostics.

Specific Methods of Operation

As described above, the reported mucus speed results of healthy subjects seem to describe a wide range of mucus flow speed. As described above, the mucus speed is affected by various parameters including environment temperature, humidity and the subject's metabolism. The mucus speed in subjects with muco-ciliary disorders is generally lower than in healthy subjects. However the speed range in healthy and disorder groups may overlap. At the time the measurements of Matsui [1] and ICRP were conducted, the researchers did not have tools to analyze the mucus flow profile in-vivo. It is assumed that this overlap diverted most of the effort in the muco-ciliary diagnosis field towards CBF based probing. However, it has been established that this parameter is not a reliable tool for detecting muco-ciliary disorders.

As described above, the system and labeling particles of the present invention provides methods for locally probing the mucus speed at a known height above the ciliary tissue. In addition, the system of the present invention provides methods for measuring the mucus speed vector, and the CBF. Some preferred methods for utilizing the system capabilities for detecting muco-ciliary disorders will be explained below.

In the study described in reference [12] the investigators conducted multiple scans of in-vivo mucus seeded with labeling particles. They extracted the discrete tracks of many labeling particles and generated an image comprising these tracks, wherein the color of each point in the track represents the local particle speed. From the generated image, one can deduce that the mucus flows in one or more well defined main streams. The flow is maximal at the center of the stream and gradually reduces as the edges. Further, the stream lines are almost parallel to each other.

The laminar speed profile of a Newtonic viscous fluid is given by the equation:

σ=μ∇\vec{V}

Where σ is the shear stress induced by the ciliary motion adjacent to the fluid at this profile point. The analysis of the image described above, seems to indicate a regular viscous flow with no induced shear stresses other than the directional ciliary motion and the viscous friction with the slower mucus streams.

Further, as described by Lasne [1], the vertical speed profile is almost constant at height above ˜100 microns. Thus, the combination of these results lead to the conclusion that the speed at such height is highly correlated with the local mucus speed averaged over the height flow profile. Further, the mucus speed measured by the system of the present invention is a reciprocal indication of the Mucus Transport Time (MTT).

According to the findings of [1-3] there may be an overlap between the mucus speed values of healthy subjects (typically 40 micron/second) and subjects with muco-ciliary disorders. In turn, using the mucus speed values as is, may lead to “false positive” diagnosis, i.e., false indication of muco-cilary disorder.

In certain aspects of the present invention, said overlap is avoided by normalizing the measured mucus speed to another value measured from the same subject. In certain aspects, the measured mucus speed values are normalized to the maximal mucus speed, for example the maximal speed may be measured at the center of main stream and near the mucus source. Low normalized speed value at certain location may indicate faulty ciliary tissue (possibly due to inflamed tissue) or highly viscous mucus.

In other aspects, the system is operated within the frame of periodical tests (e.g., occupation health tests) and the mucus speed values are normalized to the respective speed values measured at similar location on the same subject at the first tests. Systematic reduction may indicate ciliary tissue damage due to exposure to damaging contaminant.

As described above, the system of the present invention enable the probing of the muco-ciliary recovery time constant by temporary “freezing” the mucus in the FOV using a shutter. The recovery time constant is a local indication of the ciliary tissue ability to impart momentum to the adjacent mucus and it is a measure of its functionality. Such measured results can be utilized to probe ciliary tissue damage within a suspected zone (e.g., a damaged region with modified color of a smoker subject).

As described above, the system of the present invention provides methods for measuring the mucus flow vector (speed and direction). In certain aspects, the maneuverable unit of the present invention is rigid and thus the processing means can calculate the mucus speed vector in relation to a certain datum of the subject (e.g., neck axis). In certain aspects, the ciliary tissue comprises a flow block (e.g., local inflammation or a polyp) forcing the stream lines to split and reconnect around said block. A detected split or angular deviation may indicate the presence of the flow block on the ciliary tissue.

As described above, the system of the present invention provides methods for measuring the CBF. In certain aspects, the measured mucus speed is normalized to the CBF. The resulting parameter is expressed in length units (e.g., mm) and describes the coherence length between discrete cilia in the ciliary tissue. Reduced normalized values are indication of incoherent ciliary tissue or reduced MWF.

It is established that prolonged intubation is associated with bacterial infection of the lower respiratory tracts and the lungs. Prolonged intubation may be conducted in intensive care patients, elderly ventilated patients, prolonged surgery patients and children.

Ventilator-associated pneumonia (VAP) continues to be a disturbing problem in many ventilation procedures such as intensive care unit (ICU) patients. Although much progress has been achieved in the past two decades, many issues remain unresolved, and mortality still reaches 30-40%. VAP develops by a multi-stage process involves biofilm generation on the lower respiratory and lung surfaces. The biofilm protects the pneumonia bacteria against antibiotics and promote the conduction for the hosted bacteria to become antibiotics resistant.

In an animal study, Bessi et al [14] demonstrated that, following tracheal intubation, gravitational force influences tracheal mucus clearance. They found that the intubation induces a reversed ciliary motion which together with the gravitational force induces mucus flow from the proximal trachea toward the lungs. Thus instead of continuous removal of bacteria, the mucus effectively introduces bacteria into the sensitive lung surfaces. It is not surprising that when the trachea is oriented above horizontal, that flow of mucus is highly associated with bacterial colonization of the airways and pneumonia.

Bessi's finding is supported by clinical studies of some new ventilator devices which enable intermittent mucus suctioning and repeated irrigation and thus prevent contaminated mucus flow into the lungs. Still intubation for extended period is a source for various bacterial contamination

In certain aspects, the maneuverable unit of the system of the present invention equipped with a disposable removably attached unit is inserted into the lower respiratory tracts, during each event of ventilator tube replacement. Once a flow reversal is detected, the patient will be treated by a suitable treatment selected from: medication for enhancing the CBF, mucus suctioning, irrigation with suitable liquid such as saline or changing the patient position.

In other aspects, the removably attached unit of the present invention is integrated into the ventilation tube. Before replacing this ventilation tube, a suitable flexible maneuverable unit of the present invention is introduced into the ventilation tube and locked in a position suitable for detecting the mucus flow direction. When a flow reversal is detected, the ventilation tube is removed and the patient is treated with suitable treatment for restoring the normal mucus flow direction.

In some possible preferred embodiments of the invention, the system operator introduces the maneuverable unit into the nasal chamber and diagnoses pre-determined regions within the nasal chamber. In other possible preferred embodiments of the invention, the operator scans the nasal chamber tissue through a pre-determined scan pattern, preferably from anterior to posterior and from superior to inferior of the nasal chamber. In further possible preferred embodiments of the invention, a visual or audio indication will be triggered when the system diagnoses significantly slow mucus speed within the probed region. Such indication enables fast mapping of faulty ciliary tissue regions during the scan pattern.

In some possible preferred embodiments of the invention, an alarm indication may be triggered when the removably attached unit applies excessive pressure on the ciliary tissue. In some possible preferred embodiments of the invention, the pressure applied on the supporting legs is monitored by distorting a force sensitive element comprised within said flexible means attached to the maneuverable unit. The said distortion may be sensed for example by illumination of a reflective section incorporated on said force sensitive element. An optical detector (for example margin pixels in the optical sensor) may be employed for sensing the change of reflected light while the processing unit translate this change into an alarm when needed.

In some possible preferred embodiments of the invention a saline gel or solution is administered to the nose for removing or diluting the mucus. Such an operation is important in cases where the mucus is stagnant. Dilution may enable differentiating between highly viscous mucus and ineffective ciliary tissue. In other possible preferred embodiments of the invention a blurring agent may be administered to the subject, thus minimizing his uncontrolled motion and in turn reduce random motion between the removably attached unit and the tissue.

As described above, the maneuverable unit of the present invention may be used for in-vivo probing of muco-ciliary dynamics in other body lumens which comprise ciliary tissue. For each specific application, the maneuverable unit may be designed specifically for the anatomy and requirements of the specific lumen, such as Bronchi, Vaginal, etc.

In some possible preferred embodiments of the invention, the removably attached unit of the present invention is employed for localized release of beneficial substances to an accessible tissue surface. The release of substances may be conducted by methods selected from a group comprising: optically activating a pro-drug by exposure to a suitable lighting sequence, releasing a drug from a suitable dispensing means positioned on the distal end of the maneuverable unit, and applying a suitable voltage sequence on a region comprising the FOV using electrodes connected to the distal end of the maneuverable unit.

In other aspects, the removably attached unit of the present invention is utilized for drug administration onto ciliary tissue surface. The study reported in reference [13] indicates that the mucus flow impede drug delivery to a target ciliary tissue region by effective “washing away” said drug at close proximity to said target region. In addition, there have developed certain nanoparticles manufactured for preferred diffusing through the flowing mucus layer towards the treated ciliary tissue. Such nanoparticles may be locally released by the dispensing means of the present invention, as part of certain system operation modes.

In certain aspects, the removably attached unit of the present invention is utilized for testing the effectiveness of certain drug, according to the following steps: (a) Detecting a mucus flow disorder at a certain ciliary tissue region. (b) Localized delivery of the tested drug to the said probed ciliary tissue by propagating a suitable light sequence through the removably attached unit. Optionally, the localized delivery is conducted by optically activating a pro-drug previously administered to the said target region. (c) Using the system of the present invention for detecting a change in the mucus dynamics as a result of the localized drug delivery.

The systems and methods of the present invention are operable for real time probing of liquid flow close to a surface, the system comprises: A magnifying imaging optics assembly encased in an envelope, an optical imager; the removably attached unit of the present invention comprising a seeding means for localized seeding the liquid within, or at close proximity to the imaged region with at least one type of labeling particles; A modulated illumination light source operable for enhancing visibility of said labeling particles to said optical imager. The methods comprise the steps of: (a) Attaching the removably attached unit of the present invention within the liquid flowing on a desired region on said surface. (b) Releasing labeling particles adjacent or within the maneuverable unit FOV, preferably from said removably attached unit; (c) exposing said probed surface to modulated light from a suitable light source (d) capturing sufficient number of images from said probed surface region, preferably synchronized with the modulation sequence of said modulated light source. (e) extracting the flow dynamics parameter of said liquid flow.

In some possible preferred embodiments of the invention, the removably attached unit of the present invention is implemented as lab-on-a-chip devices comprising an input port into which the tested liquid is placed and channels through which said liquid flows. An external coupling optics, illumination means, an electronic imager and processing means, are comprised within the instrument which reads said lab-on-a-chip device. Said liquid comprises labeling particles and may include, for example, anything which may contain an analyte, including a biological sample, such as a biological fluid or biological cells mixed with a liquid. Examples of biological fluids include urine, blood, plasma, serum, saliva, semen, stool, sputum, cerebral spinal fluid, tears, mucus, amniotic fluid or the like.

The methods of the present invention may be used for low cost ex-vivo probing of muco-ciliary dynamics. For example, the ciliary tissue sample may be removed as nasal smear and placed on a surface in an environment suitable for preserving the ciliary tissue sample viability.

A preferred method for ex-vivo probing of muco-ciliary dynamics comprises the steps of: (a) Taking a ciliary tissue sample from proper region within a desired organ surface and placing it on a surface and providing environment suitable for preserving said sample viability; (b) Attaching the removably attached unit of the present invention on a desired region on said ciliary tissue sample. (c) Releasing labeling particles adjacent or within the FOV of said removably attached unit, preferably from wells disposed in said removably attached unit; (d) capturing sufficient number of FOV images for extracting muco-ciliary dynamics information from said ciliary tissue sample.

All of the abovementioned parameters are given by way of example only, and may be changed in accordance with the differing requirements of the various embodiments of the present invention. Thus, the abovementioned parameters should not be construed as limiting the scope of the present invention in any way. In addition, it is to be appreciated that the different tubes, optical means, and other members, described hereinabove may be constructed in different shapes (e.g. having oval, square etc. form in plan view) and sizes differing from those exemplified in the preceding description.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

### REFERENCE LIST

[1] H. Matsui, S. H. Randell et. al., “Coordinated Clearance of Periciliary Liquid and Mucus from Airway Surfaces,” J. Clin. Invest. V 102, n6 p. 1125-1131 (1998).

[2] D. J. Smith, E. A. Gaffney, et. al., “A Viscoelastic Traction Layer Model of Muco-Ciliary Transport,” Bull. Math. Biol. V 69: p. 289-327 (2007).

[3] International Commission on Radiological Protection and Measurements, Human respiratory tract model for radiological protection—ICRP Publication 66. Ann. ICRP V 24, p. 1- (1994).

[4] D. Lasne, A. Maali et. al., “Velocity Profiles of Water Flowing Past Solid Glass Surfaces Using Fluorescent Nanoparticles and Molecules as Velocity Probes” Phys. Rev. Lett. V 100, p. 214502-214504 (2008).

[5] F. C. Cheong, B. Sun, et. al., “Flow visualization and flow cytometry with holographic video microscopy,” Optics Express 1V 17 p. 13071-13079 (2009)].

[6] X. Xu, J. Luo et. al., “A PIV system for two-phase flow with nanoparticles,” Int. J. Surface Science and Eng., V 2 n1/2 (2008).

[7] Y-R. Chang, H-Y. Lee et. al., “Mass production and dynamic imaging of fluorescent nanodiamonds”, Nature Nanotech. V 3 p. 284-288 (2008)].

[8] C-C Fu, H-Y Lee et. al., “Characterization and application of single fluorescent nanodiamonds as cellular biomarkers,” PNAS V 104 n3 p. 727-732 (2007).

[9] Toshiba publication: Introducing nanoparticles using SOI chip (2006).

[10] Y-Y Hui, C-L Cheng et. al., “Nanodiamonds for optical bioimaging,” J. Phys. D: Appl. Phys. V 43 p. 374021-374031 (2010).

[11] T. Vladkova, “Surface engineering for non-toxic biofouling control (Review),” J. Univ. Chem. Technol. & Metallurg V 42 n3, 2007 p. 239-256 (2007).

[12] Y. Cu and W. M. Saltzman “Drug delivery: Stealth particles give mucus the slip,” Nature Materials V 8 p. 11-13 (2009).

[13] University of North Carolina bulletin, “Mucus flow visualization”, CISMM (Oct. 7, 2009).

[14] G. K. Bassi, A. Zanella et al. “Following tracheal intubation, mucus flow is reversed in the semirecumbent position: possible role in the pathogenesis of ventilator-associated pneumonia,” Crit Care Med V 36 p. 518-525 (2008).

1. A system for measuring the direction and speed of movement of mucus flowing along a ciliary tissue surface, wherein said system comprises:
a) a probing unit comprising:
dispensing means for controlled seeding of labeled particles into said flowing mucus;
probe illumination means for illuminating the mucus flowing over said ciliary tissue surface;
optical sensing means for detecting the movement of said labeled particles;
optical coupling means for optically coupling said illuminated mucus to said optical sensing means; and
b) a control unit comprising at least one illumination source, and means for processing optical or electrical signals received from said optical sensing means and determining the direction and speed of said mucus according to said received signals;
c) means for transferring optical or electrical or control signals between said probing unit and said control unit.

2. The system according to claim 1, wherein the optical sensing means is an optical sensor.

3. The system according to claim 2, wherein the optical sensor is an electronic imager.

4. The system according to claim 1, further comprising optical light guiding means for guiding the light from the illumination source to the probe illumination means.

5. The system according to claim 4, wherein the optical light guiding means are fiber optics.

6. The system according to claim 1, wherein said labeling particles seeded by said dispensing means are selected from the group consisting of: fluorescent nanoparticles, colored particles, directionally reflecting particles, reflecting metal particles and substances which enhance the visibility of natural particles within the mucus to said optical sensing means.

7. The system according to claim 3, wherein the signals from the electronic imager are processed by reconstructing the individual tracks of at least a portion of the labeling particles thus determining the direction and speed of the mucus.

8. The system according to claim 3, wherein the signals from the electronic imager are processed by reconstructing images of a group of labeling particles on the mucus and thus determining the direction and speed of the mucus according to the migrating of the weight center of said group.

9. A system according to claim 3, wherein the optical coupling means, the electronic imager, the light guiding means, and the means for transferring optical or electrical or control signals are comprised in a maneuverable unit comprising long slender envelope, a distal end, a proximal end; and a handle attached to the proximal end of said elongated envelope.

10. The system according to claim 9, wherein the maneuverable unit comprises two imaging channels, one channel comprising magnifying imaging optics for navigating the maneuverable unit towards the probed ciliary tissue, and the second channel comprises the optical coupling means and optical sensing means for detecting the movement of the labeled particles.

11. The system according to claim 9, wherein said maneuverable unit further comprises: a dispensing means for localized seeding the mucus within, or at close proximity to the imaged region with at least one type of labeling particles; and the probe illumination means;

12. The system according to claim 1, wherein the probing unit is divided into two distinct units:
a) the maneuverable unit comprising:
light guiding means for guiding light from the light source in the control and analysis unit to the maneuverable unit distal end;
optical imager for detecting the movement of said labeled particles;
optical coupling means for optically coupling said illuminated mucus to said optical sensing means;
b) a removably attached probe unit comprising
dispensing means for controlled seeding of labeled particles into said flowing mucus;
probe illumination means for illuminating the mucus flowing over said ciliary tissue surface;
wherein said removably attached probe comprises a view port, and wherein said probe illumination means comprises at least one illumination port, and at least one minor at large angle to said view port axis, operable for folding the light emitted from the light guiding means and passing through the illumination port, at an angle substantially perpendicular to said view port axis.

13. The system according to claim 9, wherein said maneuverable unit further comprises a filter placed between the optical coupling means and the electronic imager, operable for enhancing the labeling particles contrast vs. background light reaching from the illuminated mucus.

14. The system according to claim 13, wherein the labeling particles are fluorescent particles.

15. The system according to claim 13, wherein the filter is a dichroic filter.

16. The system according to claim 1, wherein the illumination source is a one modulated laser source.

17. The system according to claim 16 wherein the modulated laser source operates at wavelengths selected from the group consisting of: 420 nm violet diode laser, 473 nm laser, diode pumped 532 nm laser, 650 nm diode laser, and 780 nm diode laser.

18. The system according to claim 1 wherein said system means for processing optical or electrical signals received from said optical sensing means further determine the modulation of flow speed induced by the CBF.

19. The system according to claim 1 wherein the probing unit further comprises a removably attached unit, wherein said removably attached unit is operable for illuminating a mucus stratum at close proximity to the probed ciliary tissue surface, in such way that the CBF modulation of the mucus speed can be also extracted.

20. The system according to claim 18, wherein the means for processing optical or electrical signals received from said optical sensing means further determine the ratio between the flow speed and the CBF in length units.

21. The system according to claim 9, further comprising a flexible element installed on the maneuverable unit and adapted for minimizing motion between the probing unit and the probed ciliary tissue surface.

22. The system according to claim 21 wherein the flexible element is a toroidal balloon held with its axis substantially parallel to the probing element axis.

23. The system according to claim 22, wherein said balloon further comprises at least one labeling particles dispensing means, and electrical leads operable for controlled dispensing of said labeling particles.

24. The system according to claim 1, wherein the means for delivering optical or electrical signals between said probing unit and said control and analysis unit are electrical cables.

25. The system according to claim 1 wherein the means for processing optical or electrical signals received from said optical sensor and determining the direction and speed of said mucus according to said received signals is a CPU.

26. The system according to claim 10 wherein the control unit comprises an additional illumination source, and wherein said maneuverable unit comprises a separate optical light guiding means for guiding the light from said illumination source to said first channel.

27. The system according to claim 1, wherein the dispensing means comprise a well filled with labeling particles, an electronic driven piston and a dispensing cup.

28. A system according to claim 1, wherein
the dispensing means comprise a well filled with labeling particles, an electronic driven piston and a dispensing cup;
the optical sensing means is an electronic imager;
the optical coupling means is imaging optics;
the illumination source is a one modulated laser source;
the means for processing optical or electrical signals received from said electronic imager and determining the direction and speed of said mucus according to said received signals, is a CPU; and
the means for transferring optical or electrical or control signals between said probing unit and said control unit are electric cables.

29. A method of performing real-time analysis of mucus flowing on a tissue surface in a subject body, comprising the steps of:
(a) seeding the mucus above said tissue comprising ciliary structures with a sufficient number of labeling particles;
(b) positioning the maneuverable unit of claim 9, wherein the imaging optics focal plane is preferably at close proximity to said surface region wherein said imaging optics images said focal plane onto an optical sensor;
(c) exposing said surface region to a time varying illumination suitable for enhancing visibility said labeling particles to said electronic imager;
(d) capturing multiple signal arrays sets from said optical imager, preferably captured synchronously with said time varying illumination;
(e) processing said multiple signal arrays to extract movement history of at least a portion of said imaged labeling particles so as to extract the mucus flow speed and direction from said movement history.

30. A method according to claim 29, for operating a system for measuring the direction and speed of movement of mucus flowing along a ciliary tissue surface, wherein said system comprises:
a probing unit comprising:
dispensing means for controlled seeding of labeled particles into said flowing mucus:
probe illumination means for illuminating the mucus flowing over said ciliary tissue surface;
optical sensing means for detecting the movement of said labeled particles;
optical coupling means for optically coupling said illuminated mucus to said optical sensing means; and
a control unit comprising at least one illumination source, and means for processing optical or electrical signals received from said optical sensing means and determining the direction and speed of said mucus according to said received signals;
means for transferring optical or electrical or control signals between said probing unit and said control unit; and
wherein the probing unit is divided into two distinct units:
the maneuverable unit comprising:
light guiding means for guiding light from the light source in the control and analysis unit to the maneuverable unit distal end;
optical imager for detecting the movement of said labeled particles;
optical coupling means for optically coupling said illuminated mucus to said optical sensing means;
a removably attached probe unit comprising:
dispensing means for controlled seeding of labeled particles into said flowing mucus;
probe illumination means for illuminating the mucus flowing over said ciliary tissue surface;
wherein said removably attached probe comprises a view port, and wherein said probe illumination means comprises at least one illumination port, and at least one mirror at large angle to said view port axis, operable for folding the light emitted from the light guiding means and passing through the illumination port, at an angle substantially perpendicular to said view port axis, and
wherein said labeling particles are scattering particles, said illumination light is coherent light, the method further comprising the steps of:
(a) seeding a small liquid region with scattering labeling particles;
(b) positioning the maneuverable unit with the removably attached unit at close proximity to said liquid region;
(c) exposing said surface region to modulated laser illumination through the folding mirror of the removably attached unit, operable for inducing holographic pattern around each of said scattering labeling particles;
(d) capturing multiple signal arrays of said exposed surface region using said optical imager, preferably captured synchronously with said laser modulation;
(e) processing said multiple frames to identify the loci of interference patterns induce by said scattering particles; and
(f) using said loci for reconstructing the tracks of at least a portion of said scattering labeling particles so as to extract the liquid flow parameters from said tracks.

31. A system according to claim 9, wherein the maneuverable unit further comprises a flexible element attached to the long slender envelope.

32. A method for operating the system of claim 28, wherein said method comprises the steps of:
(a) inserting the distal end of the maneuverable unit towards a location of a ciliary tissue to be probed;
(b) locating the maneuverable unit at close proximity to the desired ciliary tissue region;
(c) stabilizing the maneuverable unit distal end to the ciliary tissue by releasing said flexible element;
(d) locally seeding the mucus layer above said ciliary tissue with labeling particles;
(e) exposing said mucus region to said modulated light and capture images of said mucus region in synchronization with said modulated light sequence;
(f) processing a set of captured image for extracting the desired mucus speed and direction;

33. A method according to claim 29, wherein the ciliary tissue is located on an organ selected from a group consisting of the upper respiratory system, the lower respiratory system, the female reproduction system the eye and brain.

34. A system for probing flow dynamics of a liquid flowing on a surface within a small localized region at close proximity to a surface, comprising: a magnifying imaging optics assembly encased in an envelope, an optical imager; a seeding assembly for localized seeding the liquid within, or at close proximity to the imaged region with at least one type of labeling particles; a modulated illumination light source operable for enhancing detection of said labeling particles; means for leading said illumination light to the probed surface region; means for exporting said imager data; means for receiving and processing said imager data for calculating said liquid flow dynamics parameters, wherein said means for leading the illuminating light comprise at least one tilted mirror which deflect said illumination light to a direction substantially parallel to said surface.
